SEQUENCE LISTING (86 sequences)

SEQ ID NO: 1 - protein, 22 aa, synthetic construct
  REGION 1..22: note = Signal Sequence
MDMRVPAQLLGLLLLWLPGARC

SEQ ID NO: 2 - protein, 303 aa, synthetic construct
  REGION 1..303: note = INS001
FVNQHLCGSHLVEALYLVCGERGFFYTPKTEEYPGDVGIVEQCCTSICSLEQLENYCGGEAEDLQVGQVELGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 3 - protein, 320 aa, synthetic construct
  REGION 1..320: note = INS002
FVNQHLCGSHLVEALYLVCGERGFFYTDKTEEYPGDVGIVEQCCTSICSLEQLENYCGGEAEDLQVGQVELGGGPGAGSLQPLALEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 4 - protein, 318 aa, synthetic construct
  REGION 1..318: note = INS003
SFVNQHLCGSHLVEALYLVCGERGFFYTPKTEEYPGDVGIVEQCCTSICSLEQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 5 - protein, 318 aa, synthetic construct
  REGION 1..318: note = INS004
SFVNQHLCGSHLVEALYLVCGERGFFYTPKTGSYPGGVGIVEQCCTSICSLEQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 6 - protein, 318 aa, synthetic construct
  REGION 1..318: note = INS005
SFVNQHLCGSHLVEALHLVCGERGFAYTDKTEEYPGDVGIVEQCCTSICSLEQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 7 - protein, 318 aa, synthetic construct
  REGION 1..318: note = INS006
SFVNQHLCGSHLVEALELVCGERGFHYTDKTEEYPGDVGIVEQCCTSICSLEQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 8 - protein, 318 aa, synthetic construct
  REGION 1..318: note = INS007
SFVNQHLCGSHLVEALHLVCGERGFAYTDKTGSYPGGVGIVEQCCTSICSLEQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 9 - protein, 318 aa, synthetic construct
  REGION 1..318: note = INS008
SFVNQHLCGSHLVEALELVCGERGFHYTDKTGSYPGGVGIVEQCCTSICSLEQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 10 - protein, 318 aa, synthetic construct
  REGION 1..318: note = INS009
SFVNQHLCGSHLVEALELVCGERGFHYTPKTGSYPGGVGIVEQCCTSICSLEQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 11 - protein, 318 aa, synthetic construct
  REGION 1..318: note = INS010
SFVNQHLCGSHLVEALHLVCGERGFAYTPKTEEYPGDVGIVEQCCTSICSLEQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 12 - protein, 318 aa, synthetic construct
  REGION 1..318: note = INS011
SFVNQHLCGSHLVEALHLVCGERGFAYTPKTGSYPGGVGIVEQCCTSICSLEQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 13 - protein, 317 aa, synthetic construct
  REGION 1..317: note = INS012
FVNQHLCGSHLVEALHLVCGERGFHYTPKTEEYPGDVGIVEQCCTSICSLSQLEDYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 14 - protein, 317 aa, synthetic construct
  REGION 1..317: note = INS013
FVNQHLCGSHLVEALHLVCGERGFHYTPKTGSYPGGVGIVEQCCTSICSLSQLEDYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 15 - protein, 318 aa, synthetic construct
  REGION 1..318: note = INS014
SFVNQHLCGSHLVEALHLVCGERGFHYTPKTGSYPGGVGIVEQCCTSICSLSQLEDYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 16 - protein, 318 aa, synthetic construct
  REGION 1..318: note = INS015
SFVNQHLCGSHLVEALHLVCGERGFHYTPKTEEYPGDVGIVEQCCTSICSLSQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 17 - protein, 317 aa, synthetic construct
  REGION 1..317: note = INS016
FVNQHLCGSHLVEALHLVCGERGFHYTPKTGSYPGGVGIVEQCCTSICSLSQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 18 - protein, 318 aa, synthetic construct
  REGION 1..318: note = INS017
SFVNQHLCGSHLVEALHLVCGERGFHYTPKTGSYPGGVGIVEQCCTSICSLSQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 19 - protein, 317 aa, synthetic construct
  REGION 1..317: note = INS018
FVNQHLCGSHLVEALELVCGERGFHYTPKTGSYPGGVGIVEQCCTSICSLSQLEDYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 20 - protein, 318 aa, synthetic construct
  REGION 1..318: note = INS019
SFVNQHLCGSHLVEALELVCGERGFHYTPKTGSYPGGVGIVEQCCTSICSLSQLEDYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 21 - protein, 317 aa, synthetic construct
  REGION 1..317: note = INS020
FVNQHLCGSHLVEALELVCGERGFHYTPKTEEYPGDVGIVEQCCTSICSLSQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 22 - protein, 320 aa, synthetic construct
  REGION 1..320: note = INS021
SFVNQHLCGSHLVEALELVCGERGFHYTPKTEEYPGDVGIVEQCCTSICSLSQLENYCGGEAEDLQVGQVELGGGPGAGSLQLALEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 23 - protein, 317 aa, synthetic construct
  REGION 1..317: note = INS022
FVNQHLCGSHLVEALELVCGERGFHYTPKTGSYPGGVGIVEQCCTSICSLSQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 24 - protein, 318 aa, synthetic construct
  REGION 1..318: note = INS023
SFVNQHLCGSHLVEALELVCGERGFHYTPKTGSYPGGVGIVEQCCTSICSLSQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 25 - protein, 314 aa, synthetic construct
  REGION 1..314: note = INS024
SFVNQHLCGSHLVEALELVCGERGFHYTPKTGSYPGGVGIVEQCCTSICSLEQLENYCGGEAEDLQVGQVELGGGPGAGSLQPEGSLQDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 7 aa, synthetic construct
  REGION 1..7: note = Linker 001
GSYPGGV

SEQ ID NO: 27 - protein, 7 aa, synthetic construct
  REGION 1..7: note = Linker 002
EEYPGDV

SEQ ID NO: 28 - protein, 14 aa, synthetic construct
  REGION 1..14: note = Linker 003
EAEDLQVGQVELGG

SEQ ID NO: 29 - protein, 29 aa, synthetic construct
  REGION 1..29: note = Linker 004
GEAEDLQVGQVELGGGPGAGSLQPEGSLQ

SEQ ID NO: 30 - protein, 110 aa, Homo sapiens
  REGION 1..110: note = INS_HUMAN Insulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 31 - protein, 31 aa, Homo sapiens
  REGION 1..31: note = INS_HUMAN C-PEPTIDE
EAEDLQVGQVELGGGPGAGSLQPLALEGSLQ

SEQ ID NO: 32 - protein, 30 aa, Homo sapiens
  REGION 1..30: note = Insulin B-chain human
FVNQHLCGSHLVEALYLVCGERGFFYTPKT

SEQ ID NO: 33 - protein, 21 aa, Homo sapiens
  REGION 1..21: note = Insulin A-chain human
GIVEQCCTSICSLYQLENYCN

SEQ ID NO: 34 - protein, 211 aa, Homo sapiens
  REGION 1..211: note = IGHG1
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 330 aa, Homo sapiens [sequence omitted]

SEQ ID NO: 36 - protein, 12 aa, Homo sapiens
  REGION 1..12: note = IGHG1 hinge
EPKSCDKTHTCP

SEQ ID NO: 37 - protein, 226 aa, Homo sapiens
  REGION 1..226: note = IGHG1
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 327 aa, Homo sapiens
  REGION 1..327: note = IGHG4_HUMAN
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 12 aa, Homo sapiens
  REGION 1..12: note = IGHG4 hinge
ESKYGPPCPSCP

SEQ ID NO: 40 - protein, 13 aa, Homo sapiens
  REGION 1..13: note = IGHG4
ESKYGPPCPSCPA

SEQ ID NO: 41 - protein, 13 aa, Homo sapiens
  REGION 1..13: note = IGHG4 S108P
ESKYGPPCPPCPA

SEQ ID NO: 42 - protein, 6 aa, synthetic construct
  REGION 1..6: note = Linker 005
GGGGSA

SEQ ID NO: 43 - protein, 58 aa, synthetic construct
  REGION 1..58: note = Sequence
FVNQHLCGSHLVEALYLVCGERGFFYTPKTEEYPGDVGIVEQCCTSICSLEQLENYCG

SEQ ID NO: 44 - protein, 58 aa, synthetic construct
  REGION 1..58: note = Sequence
FVNQHLCGSHLVEALYLVCGERGFFYTDKTEEYPGDVGIVEQCCTSICSLEQLENYCG

SEQ ID NO: 45 - protein, 59 aa, synthetic construct
  REGION 1..59: note = Ins505
SFVNQHLCGSHLVEALYLVCGERGFFYTPKTEEYPGDVGIVEQCCTSICSLEQLENYCG

SEQ ID NO: 46 - protein, 59 aa, synthetic construct
  REGION 1..59: note = Ins754
SFVNQHLCGSHLVEALHLVCGERGFAYTDKTEEYPGDVGIVEQCCTSICSLEQLENYCG

SEQ ID NO: 47 - protein, 59 aa, synthetic construct
  REGION 1..59: note = Ins756
SFVNQHLCGSHLVEALELVCGERGFHYTDKTEEYPGDVGIVEQCCTSICSLEQLENYCG

SEQ ID NO: 48 - protein, 59 aa, synthetic construct
  REGION 1..59: note = Ins759
SFVNQHLCGSHLVEALHLVCGERGFAYTDKTGSYPGGVGIVEQCCTSICSLEQLENYCG

SEQ ID NO: 49 - protein, 59 aa, synthetic construct
  REGION 1..59: note = Ins761
SFVNQHLCGSHLVEALELVCGERGFHYTDKTGSYPGGVGIVEQCCTSICSLEQLENYCG

SEQ ID NO: 50 - protein, 59 aa, synthetic construct
  REGION 1..59: note = Ins792
SFVNQHLCGSHLVEALELVCGERGFHYTPKTGSYPGGVGIVEQCCTSICSLEQLENYCG

SEQ ID NO: 51 - protein, 59 aa, synthetic construct
  REGION 1..59: note = Ins799
SFVNQHLCGSHLVEALHLVCGERGFAYTPKTEEYPGDVGIVEQCCTSICSLEQLENYCG

SEQ ID NO: 52 - protein, 59 aa, synthetic construct
  REGION 1..59: note = Ins800
SFVNQHLCGSHLVEALHLVCGERGFAYTPKTGSYPGGVGIVEQCCTSICSLEQLENYCG

SEQ ID NO: 53 - protein, 58 aa, synthetic construct
  REGION 1..58: note = Ins805
FVNQHLCGSHLVEALHLVCGERGFHYTPKTGSYPGGVGIVEQCCTSICSLSQLEDYCG

SEQ ID NO: 54 - protein, 59 aa, synthetic construct
  REGION 1..59: note = Ins807
SFVNQHLCGSHLVEALHLVCGERGFHYTPKTGSYPGGVGIVEQCCTSICSLSQLEDYCG

SEQ ID NO: 55 - protein, 59 aa, synthetic construct
  REGION 1..59: note = Ins811
SFVNQHLCGSHLVEALHLVCGERGFHYTPKTEEYPGDVGIVEQCCTSICSLSQLENYCG

SEQ ID NO: 56 - protein, 58 aa, synthetic construct
  REGION 1..58: note = Ins813
FVNQHLCGSHLVEALHLVCGERGFHYTPKTGSYPGGVGIVEQCCTSICSLSQLENYCG

SEQ ID NO: 57 - protein, 59 aa, synthetic construct
  REGION 1..59: note = Ins815
SFVNQHLCGSHLVEALHLVCGERGFHYTPKTGSYPGGVGIVEQCCTSICSLSQLENYCG

SEQ ID NO: 58 - protein, 58 aa, synthetic construct
  REGION 1..58: note = Ins835
FVNQHLCGSHLVEALELVCGERGFHYTPKTGSYPGGVGIVEQCCTSICSLSQLEDYCG

SEQ ID NO: 59 - protein, 59 aa, synthetic construct
  REGION 1..59: note = Ins837
SFVNQHLCGSHLVEALELVCGERGFHYTPKTGSYPGGVGIVEQCCTSICSLSQLEDYCG

SEQ ID NO: 60 - protein, 58 aa, synthetic construct
  REGION 1..58: note = Ins839
FVNQHLCGSHLVEALELVCGERGFHYTPKTEEYPGDVGIVEQCCTSICSLSQLENYCG

SEQ ID NO: 61 - protein, 59 aa, synthetic construct
  REGION 1..59: note = Ins842
SFVNQHLCGSHLVEALELVCGERGFHYTPKTEEYPGDVGIVEQCCTSICSLSQLENYCG

SEQ ID NO: 62 - protein, 58 aa, synthetic construct
  REGION 1..58: note = Ins843
FVNQHLCGSHLVEALELVCGERGFHYTPKTGSYPGGVGIVEQCCTSICSLSQLENYCG

SEQ ID NO: 63 - protein, 59 aa, synthetic construct
  REGION 1..59: note = Ins845
SFVNQHLCGSHLVEALELVCGERGFHYTPKTGSYPGGVGIVEQCCTSICSLSQLENYCG

SEQ ID NO: 64 - protein, 30 aa, synthetic construct
  REGION 1..30: note = InsB-AspB28
FVNQHLCGSHLVEALYLVCGERGFFYTDKT

SEQ ID NO: 65 - protein, 31 aa, synthetic construct
  REGION 1..31: note = InsB-SerB0
SFVNQHLCGSHLVEALYLVCGERGFFYTPKT

SEQ ID NO: 66 - protein, 31 aa, synthetic construct
  REGION 1..31: note = InsB-SerB0_HisB16_AlaB25_AspB28
SFVNQHLCGSHLVEALHLVCGERGFAYTDKT

SEQ ID NO: 67 - protein, 31 aa, synthetic construct
  REGION 1..31: note = InsB-SerB0_GluB16_HisB25_AspB28
SFVNQHLCGSHLVEALELVCGERGFHYTDKT

SEQ ID NO: 68 - protein, 31 aa, synthetic construct; identical to SEQ ID NO: 66
  REGION 1..31: note = InsB-SerB0_HisB16_AlaB25_AspB28

SEQ ID NO: 69 - protein, 31 aa, synthetic construct
  REGION 1..31: note = InsB-SerB0_GluB16_HisB25
SFVNQHLCGSHLVEALELVCGERGFHYTPKT

SEQ ID NO: 70 - protein, 31 aa, synthetic construct
  REGION 1..31: note = InsB-SerB0_HisB16_AlaB25
SFVNQHLCGSHLVEALHLVCGERGFAYTPKT

SEQ ID NO: 71 - protein, 30 aa, synthetic construct
  REGION 1..30: note = InsB-HisB16_HisB25
FVNQHLCGSHLVEALHLVCGERGFHYTPKT

SEQ ID NO: 72 - protein, 30 aa, synthetic construct
  REGION 1..30: note = InsB-GluB16_HisB25
FVNQHLCGSHLVEALELVCGERGFHYTPKT

SEQ ID NO: 73 - protein, 31 aa, synthetic construct; identical to SEQ ID NO: 69
  REGION 1..31: note = InsB-SerB0_GluB16_HisB25

SEQ ID NO: 74 - protein, 21 aa, synthetic construct
  REGION 1..21: note = InsA-GluA14_GlyA21
GIVEQCCTSICSLEQLENYCG

SEQ ID NO: 75 - protein, 21 aa, synthetic construct
  REGION 1..21: note = InsA-SerA14_AspA18_GlyA21
GIVEQCCTSICSLSQLEDYCG

SEQ ID NO: 76 - protein, 21 aa, synthetic construct
  REGION 1..21: note = InsA-SerA14_GlyA21
GIVEQCCTSICSLSQLENYCG

SEQ ID NO: 77 - protein, 299 aa, synthetic construct
  REGION 1..299: note = INS025
FVNQHLCGSHLVEALELVCGERGFHYGGGGGGSGGGGGIVEQCCTSTCSLDQLENYCGGGGGQGGGGQGGGGQGGGGGECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 78 - protein, 317 aa, synthetic construct
  REGION 1..317: note = INS026
FVNQHLCGSHLVEALELVCGERGFHYGGGGGGSGGGGGIVEQCCTSTCSLDQLENYCGGEAEDLQVGQVELGGGPGAGSLQPEGSLQGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 79 - protein, 308 aa, synthetic construct
  REGION 1..308: note = INS027
FVNQHLCGSHLVEALELVCGERGFHYGGGGGGSGGGGGIVEQCCTSTCSLDQLENYCGGGGGQGGGGQGGGGQGGGGGGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 80 - protein, 58 aa, synthetic construct
  REGION 1..58: note = Ins801
FVNQHLCGSHLVEALHLVCGERGFHYTPKTEEYPGDVGIVEQCCTSICSLSQLEDYCG

SEQ ID NO: 81 - protein, 59 aa, synthetic construct
  REGION 1..59: note = Single chain insulin
SFVNQHLCGSHLVEALYLVCGERGFFYTPKTGSYPGGVGIVEQCCTSICSLEQLENYCG

SEQ ID NO: 82 - protein, 59 aa, synthetic construct; identical to SEQ ID NO: 46
  REGION 1..59: note = Single chain insulin

SEQ ID NO: 83 - protein, 230 aa, synthetic construct
  REGION 1..230: note = Fc region with third linker and C-terminal hinge
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAESKYGPPCPPCPA

SEQ ID NO: 84 - protein, 225 aa, synthetic construct
  REGION 1..225: note = Fc region with third linker and C-terminal hinge
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAESKYGPPCPPCPA

SEQ ID NO: 85 - protein, 220 aa, synthetic construct
  REGION 1..220: note = Fc region with third linker and C-terminal hinge
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGPPCPPCPA

SEQ ID NO: 86 - protein, 31 aa, synthetic construct
  REGION 1..31: note = B chain insulin
SFVNQHLCGSHLVEALHLVCGERGFHYTPKT

(19) United States
(12) Patent Application Publication
(10) Pub. No.: US 2026/0248892 A1
(43) Pub. Date: Aug. 27, 2026

(54) Single chain insulins and Fc conjugates thereof

(71) Applicant: SANOFI, Paris (FR)

(72) Inventors: Thomas BOEHME, Frankfurt (DE); Oliver BOSCHEINEN, Frankfurt (DE); Werner DITTRICH, Frankfurt (DE); Marcus Hermann KORN, Frankfurt (DE); Thomas LANGER, Frankfurt (DE); Jens RIEDEL, Frankfurt (DE); Garima TIWARI, Frankfurt (DE); Ulrich WERNER, Frankfurt (DE)

(21) Appl. No.: 18/877,541

(22) PCT Filed: Jun. 21, 2023
(86) PCT No.: PCT/EP2023/066841

§ 371 Date: Dec. 20, 2024

(30) Foreign Application Priority Data
Jun. 23, 2022 (EP) 22315123.4

(51) Int. Cl. A61K 38/28; A61K 47/64; A61K 47/68

(52) U.S. Cl. CPC A61K 38/28; A61K 47/64; A61K 47/6811

(57) ABSTRACT

Provided herein is an insulin-Fc fusion polypeptide comprising a single-chain insulin and an Fc region polypeptide. Further provided is a conjugate of two insulin-Fc fusion polypeptides, wherein the two insulin-Fc fusion polypeptides are linked by at least one covalent bond at the C-terminus of the fusion polypeptides. Also provided is a single chain insulin having reduced binding affinity to the insulin receptor. Further provided is the use of the insulin-Fc fusion polypeptide, the conjugate, or the single chain insulin in medicine.

### FIELD

Provided herein is an insulin-Fc fusion polypeptide comprising a single-chain insulin and an Fc region polypeptide. Further provided is a conjugate of two insulin-Fc fusion polypeptides, wherein the two insulin-Fc fusion polypeptides are linked by at least one covalent bond at the C-terminus of the fusion polypeptides. Also provided is a single chain insulin having reduced binding affinity to the insulin receptor. Further provided is the use of the insulin-Fc fusion polypeptide, the conjugate, or the single chain insulin in medicine.

### BACKGROUND

Diabetes mellitus (DM) is a chronic metabolic disease caused by insulin deficiency, insulin resistance or both. There are two main types of DM, referred as type 1 (T1DM) and type 2 diabetes (T2DM) In T1DM the insulin-producing pancreatic beta cells have been destroyed by the immune system and hence T1DM can be considered as an autoimmune disease. As a consequence thereof, patients with T1DM are not able to produce insulin and are absolutely dependent on the administration of insulin. T2DM is a complex metabolic disease which can take years to evolve. T2DM is mostly connected with other health problem, e.g. obesity, nonalcoholic fatty liver disease and high blood pressure. The combination of the different conditions is also named as metabolic syndrome. In T2DM the pancreas is still able to produce insulin, but insulin levels may decline as the disease progresses (Sapra and Bhandari 2020; Godoy-Matos et al., 2020).

The main physiologic function of insulin is the regulation of blood glucose. Blood glucose levels are sensed by the beta cells in the pancreas and upon a certain threshold insulin is secreted in the circulation. Insulin in the circulation leads to uptake of glucose from the blood mainly into the liver, muscle and adipose tissue. Absence of insulin or insulin resistance leads to elevated blood glucose levels (hyperglycaemia) which, when untreated over a longer period of time, leads to anomalies in micro- and microvasculature which ultimately results in organ dysfunction. Most prone for perturbations of micro- and macrovasculature are the kidney, eyes, nerves and the heart (Kahn et al., 2014; Taylor et al., 2021).

The receptor for insulin (IR) is a heterotetramer consisting of two extracellular alpha-subunits and two transmembrane-spanning beta-subunits. The alpha- and beta-subunits are connected by disulfide-bonds. Within the intracellular site there is a tyrosine kinase domain. Upon insulin binding, IR dimerizes and the intracellular tyrosine-kinase domains are brought into close proximity which allows autophosphorylation and initiation of the intracellular signaling cascade. In humans, there are two isoforms of the IR present, referred as IR-A and IR-B. The difference between these isoforms is the presence of a C-terminal 12 amino acid extension of the A-subunit in the IR-B isoform. Both IR isoforms differ in their expression pattern and binding parameters for insulin as well as the insulin-like growth factors 1 and 2 (IGF1 and IGF2). For example, IR-A has a higher affinity for insulin and IGF2 than IR-B. There are also physiologic differences between IR-A and IR-B. IR-B is predominantly expressed at high levels in insulin target tissues and is assumed to mediate metabolic effects and cell growth whereas IR-A is thought to prompt cell proliferation, (Belfiore et al., 2017).

Insulin-like growth factors (IGFs) have a similar structure like insulin. Their biological function is induction of growth or cell differentiation. Signaling of IGFs is mediated by the IGF-1-receptors, which has also a similar structure as the insulin-receptor. Since ligands and receptors are similar in structure, IGF1 and IGF2 can indeed bind to and activate IR, albeit at higher concentrations. In the same manner, insulin can bind to and activate the IGF-1 receptor. The crosstalk between these signaling pathways is further entangled by the possibility of IGF-1R and IR to form functional heterodimers (Denley et al., 2007; Hakuno et Takahasi, 2018)

Insulin is synthesized by beta cells in the pancreas as a preprohormone. A signal sequence guides the secretion of insulin into the lumen of the endoplasmic reticulum (ER). During secretion the signal sequence is cleaved. The resulting proinsulin consist of three parts: A N-terminal B-chain, the connecting peptide (C-peptide) and a C-terminal A-chain (Dodson and Steiner 1998). During intracellular transport proinsulin is further processed. The C-peptide is excised and two C-terminal arginine residues at the B-chain are cut off (Steiner 2011). Mature insulin is composed of two chains (A-chain and B-chain) which are connected by two disulfide bonds.

In contrast to the mature insulin, proinsulin is a single chain protein. Proinsulin is able to fold into a three-dimensional structure although the C-peptide and the junction to the A- and B-chains are flexible (Yang et al., 2010). Proinsulin is biologic active in cellular assays (Jehle et al., 1996) and stable in human blood (Bright et al., 2017). There is evidence that proinsulin has also a physiological role during embryonic development (Hernández-Sánchez et al., 2006).

Single chain insulins (scls) are insulin variants where the Insulin B- and Insulin A-chain are connected by short peptide linkers. Such variants can easily be produced in a recombinant manner and are, depending on the length and sequence of the linker, still active. Alternatively, the insulin a- and b-chain can be connected by a chemical linker. An advantage of scls over native insulin is their improved stability (Hua et al., 1998; Glidden et al., 2018). These features make single chain insulins attractive as an alternative insulin replacement.

The aim of insulin replacement therapy is to mimic the natural insulin secretion profile. For human insulin, onset of action is ~30 min after administration and action can last for over 5 hrs. If administered in the afternoon, these properties might lead to nocturnal hypoglycaemia; hence patients are urged for a bed-time snack to avoid hypoglycaemia. In order to allow a more precise control of insulin action different insulin formulations and insulin analogues with differing pharmacokinetic properties have been developed. Insulin analogues can be classified as rapid, short, intermediate or long-acting. Rapid and short acing insulin analogs (e.g. insulin lispro, insulin aspart, insulin glulisine) can be administered shortly before or even after a meal. With long acting or basal insulin analogs (e.g. insulin detemir, insulin decludec, inulin glargine) a flatter and long-lasting inulin profile can be achieved. Long acting insulin analogs have a time of action up to 24 hr. It has turned out that with the use of long acting insulins the risk of nocturnal hypoglycaemia can be significantly be reduced (Sharma et al., 2019; Mathieu 2021). A basal insulin analogue which needs only be administered once weekly, insulin icodec, is as effective as daily administered insulin glargine for blood glucose level lowering (Rosenstock et al., 2020). Such ultra long acting insulin analogues require less frequent injections and hence can help to improve patients' quality of life.

Based on the structure, long acting insulins analogues can be classified into two groups: insulin with altered amino acid sequence (e.g. insulin glargine) and insulin analogues with chemical modifications (e.g. insulin detemir). The mode of action can be different for long acting insulin analogs. Insulin glargine has shifted isoelectric point compared to native insulin. As a consequence thereof, insulin glargine precipitates after administration and is slowly released from this depot into the blood stream. Insulin detemir is acetylated with a 14-carbon fatty acid that enables binding to albumin. Human albumin has a long plasma half-life of ~20 days. A recycling mechanism is the reason for this long half-life. The underlying recycling mechanism uses the same pathway as the recycling mechanism used by antibodies of the immunoglobulin G (IgG) class, which also have plasma half lives of ~20 days. Both, albumin and IgGs, use the recycling mechanism based on the neonatal Fc-receptor (Sand et al., 2015).

IgGs bind to the FcRn via the Fc-domain. Upon fusion or conjugation of a peptide or protein to a Fc-domain a prolonged plasma half-life for the corresponding Fc-fusion protein can be achieved and several Fc-fusion proteins have already entered the pharmaceutical market (Rath et al., 2015).

IgGs are multifunctional protein. The normal function of antibodies is to evoke the humoral and cellular immune system upon antigen-binding. While antigens are bound by Fab-domains, effector functions of antibodies are mediated by the Fc-domain. The receptors that mediate the physiological function of IgGs are called Fc-gamma receptors (FcγRs). Binding to the FcγRs activates the so-called antibody-dependent cellular cytotoxicity (ADCC) response (Bruhns and Jönsson, 2015). Another effector function of antibodies is mediated by the protein C1q. C1q belongs to a defense mechanism known as the complement system. Upon binding of C1q to antibodies a proteolytic cascade, the complement cascade is triggered. This cascade finally results in the assembly of the so-called membrane attack complex which perforates the membrane of the target cells. This immune-defense mechanism is also known as complement-dependent cytotoxicity (CDC) (West et al., 2018).

For many therapeutic antibodies, e.g. for the treatment of cancers, the effector functions are required to achieve the therapeutic goal. Indeed, increasing the potency of such antibodies can be accomplished by Fc engineering to enhance binding of the antibody to the FcγRs or C1q. The Fc-domain is glycosylated at position N297. Glycosylation of this site is required to elicit full effector function (Wang et al., 2018). On the other hand, many therapeutic antibodies and Fc-fusion proteins are designed to bind specific targets or capture unwanted, in excess present messengers like pro-inflammatory cytokines. In these cases, the Fc-mediated effector-functions are not desired; especially if the Fc-fusion protein is administered over a long period of time.

For Fc-fusion proteins it is usually not intended to compromise binding to the FcRn. In contrast to this, reduction of the effector function of the Fc-domain is desirable in certain cases. Several point mutations within the Fc-domain have been described to reduce effector function. The glycosylation site also belongs to these sites since deglycosylated antibodies or Fc-fusion proteins exhibit reduced effector functions. For Fc-fusion proteins aglycosylated proteins can be obtained done by expressing the proteins in bacteria, e.g. E. coli. However, aglycosylated variants may exhibit reduced solubility or stability (Schlothauer et al., 2016; Jacobsen et al., 2017; Dumet et al., 2019).

WO 2016/178905 A1 discloses fusion proteins comprising an insulin receptor agonist fused to a human IgG Fc region through the use of a peptide linker, and the use of such fusion proteins in the treatment of diabetes.

There remains a need for ultra long acting insulin analogs which require less frequent injections and hence can help to improve quality of life of diabetes patients.

In the studies described herein, mutations were introduced at certain positions into the Insulin B- and A chain of human insulin to generate single chain insulin analogs with reduced binding affinity for the human insulin receptor compared to human insulin. Without being bound to theory, it assumed that a reduced affinity to the insulin-receptor is beneficial for an ultra-long acting insulin since this will reduce clearance of the insulin-derivative bound to the insulin-receptor. As the activation of the IR-A receptor is sought to be responsible for the mitogenic properties of insulin, variants were produced with a more reduced activity to the IR-A than to IR-B compared with the wild type insulin. The insulins were tested in form of single chain insulin Fc fusion polypeptides. The sequences are provided in Table A in the Examples section. The results for the receptor activation are shown in Table 1 of the Examples section. Examples of such insulins with reduced activity for the IR-A compared to IR-B and ultra-long activity are INS009 (SEQ ID NO: 10), INS011 (SEQ ID NO: 12), INS013 (SEQ ID NO: 14) and INS 018 (SEQ ID NO: 19). The single chain insulin analogs present in these fusions have an amino acid sequence as shown in SEQ ID NO: 50 (INS009), SEQ ID NO: 52 (INS011), SEQ ID NO: 53 (INS014) and SEQ ID NO: 58 (INS018), respectively.

In the studies described herein, the generated single chain insulin analogs were fused to an IgG Fc-domain. Since the application of insulin occurs usually over a long period of time, an effector-less Fc-domain was chosen. In the chosen effector-less Fc-domain the hinge domain which is present at the N-terminus of the Fc region was deleted. The deletion of the hinge domain reduces the effector function, because the binding site for FcγRs and C1q, which are responsible for the antibody effector functions, are partially located in the antibody hinge region while the binding sites for FcRn and protein A are located in the more C-terminal part of the Fc region. Thus, removal of the hinge region abolishes antibody effector functions without impairing the desired Fc functions. Moreover, two C-terminal covalent bonds of the invention overcome the limitations (compromised FcRn binding, reduced stability) observed for a plain hinge-less antibody variant.

SEQ ID NO: 34 shows the amino acid sequence of an effectorless IgG1 Fc region in which the hinge region was deleted. However, a hinge region (SEQ ID NO: 41) was fused to C-terminus of the Fc region via a linker peptide (GGGGSA, SEQ ID NO: 42). Covalent linkage of the two Fc-chains was achieved by fusing the hinge sequence to the C-terminus. Advantageously, the sequence of the antibody hinge region forms inter-chain disulfide bonds also if positioned C-terminally to the Fc part. As the sequence of the antibody hinge region can be derived from a human endogenous antibody hinge region, the usage of exogeneous sequences can be avoided which reduces the risk of anti-drug antibody (ADA) formation.

A linker sequence (linker 2 in FIG. 8) connecting the single chain inulin to the Fc-domain was derived from the C-peptide, occurring in the native proinsulin. The C-peptide is a 30 amino acid long peptide (SEQ ID NO: 31) consisting mainly of small and hydrophilic amino acids. It is stable in blood and probably has a chaperone-like effect toward insulin (Landreh et al. 2013). The C-peptide in solution is not fully folded nor it has a random structure (Munte et al., 2005). Consisting of small and hydrophilic amino acids, being stable in blood and being structurally flexible predestinies the C-peptide or peptides derived from the C-peptide for usage as linker. To further increase the amount of small hydrophilic amino acids in the sequence of the second linker, amino acids Leu26Ala27Leu28 from SEQ ID NO: 31 were deleted and a Gly was placed at the N-terminus resulting in the linker sequence of SEQ ID NO: 29. The, thus, generated linker was e.g. used in the constructs designated INS003 to INS025 (see Table A).

Surprisingly, the combination of the insulin variant and the effector-less Fc-domain leads to ultra-long mode of action compared to a protein construct where the insulin variant is fused to a commonly used silenced IgG Fc-backbone. This is exemplified by the single chain insulin variant having a sequence as shown in SEQ ID NO: 50. When fused to the effectorless Fc-domain with deleted hinge (SEQ ID NO: 10, INS009) a much longer blood glucose lowering effect was observed compared to INS024 (SEQ ID NO: 25) (See FIGS. 4 and 7, and Table 2 in the Examples section). The blood glucose lowering activity for INS 009 (SEQ ID NO: 10) lasted at least for 216 hrs in the Göttingen mini pig model which was much longer than for INS024 (SEQ ID NO 25), which had a time of action for blood glucose lowering activity in the minipig of up to 120 hr. INS024 had the hinge at the normal position.

Also for the variants INS013 (SEQ ID NO: 14) and INS011 (SEQ ID NO: 12) very long blood glucose lowering activity was observed in the Göttingen minipig models, with time of actions up to 216 hrs (INS013 [SEQ ID NO: 14) (FIG. 4) and 264 hrs (INS011, SEQ ID NO: 12 (FIG. 5 and Table 2 in the Examples section).

The introduced mutations into the insulin sequence, the peptide connecting the insulin A- and B-chain (linker 2 in FIG. 8) as well as the linker sequence connecting the insulin to the Fc-domain cumulated into stable insulin-derivatives with ultra-long action in the rat model (up to 168 hr time of action) and in the minipig model (up to 264 hr time of action [INS011 [SEQ ID NO: 12])). In addition, plasma half lives up to 69 hrs were measured in cynomolgus monkeys (see Table 3 and FIG. 11).

### SUMMARY OF THE PRESENT INVENTION

Provided herein are insulin analogs with reduced affinity to the insulin receptor, such as the insulin analogs shown in Table B in the Examples section. Further provided are ultra-long acting insulin analogs comprising single chain insulins (scl) fused to an Fc-domain, advantageously to an IgG Fc-domain without an N-terminal hinge region. The scl-Fc-fusion proteins can be produced in a recombinant manner without the need for further chemical modification.

### First Aspect: Insulin Analogs

Provided herein is an insulin analog comprising an insulin B chain and an insulin A chain, wherein the insulin B chain comprises or consists of the amino acid sequence SFVNQHLCGSHLVEALELVCGERGFHYTPKT (SEQ ID NO: 73) and the insulin A chain comprises or consists of the amino acid sequence GIVEQCCTSICSLEQLENYCG (SEQ ID NO: 74).

Further provided is an insulin analog comprising an insulin B chain and an insulin A chain, wherein the insulin B chain comprises or consists of the amino acid sequence SFVNQHLCGSHLVEALHLVCGERGFAYTPKT (SEQ ID NO: 70) and the insulin A chain comprises or consists of the amino acid sequence GIVEQCCTSICSLEQLENYCG (SEQ ID NO: 74).

Further provided is an insulin analog comprising an insulin B chain and an insulin A chain the insulin B chain, wherein the insulin B chain comprises or consists of the amino acid sequence FVNQHLCGSHLVEALHLVCGERGFHYTPKT (SEQ ID NO: 71) and the insulin A chain comprises or consists of the amino acid sequence GIVEQCCTSICSLSQLEDYCG (SEQ ID NO: 75).

Further provided is an insulin analog comprising an insulin B chain and an insulin A chain the insulin B chain, wherein the insulin B chain comprises or consists of the amino acid sequence SFVNQHLCGSHLVEALELVCGERGFHYTPKT (SEQ ID NO: 73) and the insulin A chain comprises or consists of the amino acid sequence GIVEQCCTSICSLSQLEDYCG (SEQ ID NO: 75).

Further provided is an insulin analog comprising an insulin B chain and an insulin A chain, wherein the insulin B chain comprises or consists of the amino acid sequence the insulin B chain comprises or consists of the amino acid sequence SFVNQHLCGSHLVEALYLVCGERGFFYTPKT (SEQ ID NO: 65) and wherein the insulin A chain comprises or consists of the amino acid sequence GIVEQCCTSICSLEQLENYCG (SEQ ID NO: 74).

Further provided is an insulin analog comprising an insulin B chain and an insulin A chain the insulin B chain, wherein the insulin B chain comprises or consists of the amino acid sequence SFVNQHLCGSHLVEALHLVCGERGFAYTDKT (SEQ ID NO: 66) and wherein the insulin A chain comprises or consists of the amino acid sequence GIVEQCCTSICSLEQLENYCG (SEQ ID NO: 74).

Further provided is an insulin analog comprising an insulin B chain and an insulin A chain the insulin B chain, wherein the insulin B chain comprises or consists of the amino acid sequence SFVNQHLCGSHLVEALHLVCGERGFAYTDKT (SEQ ID NO: 67) and wherein the insulin A chain comprises or consists of the amino acid sequence GIVEQCCTSICSLEQLENYCG (SEQ ID NO: 74).

Typically, the insulin analogs are provided as single chain insulins. In the single chain insulin, the insulin B chain is, typically, linked to the insulin A chain via a first linker peptide.

### Second Aspect: Insulin-Fc Fusion Polypeptides and Conjugates of Two Insulin-Fc Fusion Polypeptides

Further provided is an insulin-Fc fusion polypeptide comprising from N- to C-terminus: an insulin and an Fc region polypeptide (herein also referred to as “Fc region”).

Typically, the insulin present in the fusion protein is an insulin analog having reduced binding affinity to the human insulin receptor as compared to human insulin. In an embodiment, the insulin is a single chain insulin, such as a single-chain insulin provided herein (see e.g. Table B).

Typically, the single chain insulin is linked to the Fc region polypeptide via a second linker peptide.

Accordingly, said insulin-Fc fusion polypeptide, typically, comprises from N- to C-terminus:
a) an insulin B chain,
b) a first linker peptide,
c) an insulin A chain,
d) a second linker peptide, and
e) an Fc region polypeptide

In some embodiments, the insulin-Fc fusion polypeptide further comprises
f) a third linker peptide,
g) a C-terminal peptide.

Said C-terminal peptide shall allow the formation of at least two covalent bonds between two insulin-Fc fusion polypeptides, thereby stabilizing the conjugate provided herein.

Typically, the C-terminal peptide comprises least two cysteine residues allowing the formation of at least two disulfide bonds between the two insulin-Fc fusion polypeptides present in the conjugate.

In an embodiment, the C-terminal peptide comprises the amino acid sequence of an antibody hinge region (or a highly similar sequence) comprising at least two cysteine residues allowing the formation of at least two disulfide bonds between the two insulin-Fc fusion polypeptides.

In some embodiments, the C-terminal peptide comprises or consists of an amino acid sequence as shown in SEQ ID NO: 36, 39, 40 or 41.

In some embodiments, the Fc region polypeptide comprises the constant domains of the constant documents of an IgG, IgM, IgA, IgD or IgE antibody

In some embodiments, the Fc region polypeptide comprises the constant domains CH2 and CH3 of the antibody heavy chain an IgG antibody, such as an IgG1 antibody or an IgG4 antibody.

In some embodiments, the Fc region polypeptide comprises or consists of
a) an amino acid sequence as shown in SEQ ID NO: 34, or
b) an amino acid sequence being at least 90%, at least 91%, at least 92%, at least 93%, at least 94%, at least 95%, at least 96%, at least 97%, at least 98% or at least 99% identical to SEQ ID NO: 34.

Advantageously, the Fc region polypeptide lacks an antibody hinge region at the N-terminus. Accordingly, the two insulin-Fc fusion polypeptides are typically not covalently linked by one or more disulfide bonds between the N-terminus of each Fc region polypeptide.

In some embodiments, the insulin-Fc fusion polypeptide comprises a sequence as shown in any one of SEQ ID NO: 2 to 25, and 77 and 78. The sequences are shown in Table A in the Examples section.

In some embodiments, the insulin-Fc fusion polypeptide comprises a sequence as shown in SEQ ID NO: 4 (internal name: “INS003”, see Table A).

In some embodiments, the insulin-Fc fusion polypeptide comprises a sequence as shown in SEQ ID NO: 5 (INS004).

In some embodiments, the insulin-Fc fusion polypeptide comprises a sequence as shown in SEQ ID NO: 6 (INS005).

In some embodiments, the insulin-Fc fusion polypeptide comprises a sequence as shown in SEQ ID NO: 7 (INS006).

In some embodiments, the insulin-Fc fusion polypeptide comprises a sequence as shown in SEQ ID NO: 10 (INS009),

In some embodiments, the insulin-Fc fusion polypeptide comprises a sequence as shown in SEQ ID NO: 12 (INS011),

In some embodiments, the insulin-Fc fusion polypeptide comprises a sequence as shown in SEQ ID NO: 14 (INS013),

In some embodiments, the insulin-Fc fusion polypeptide comprises a sequence as shown in SEQ ID NO: 19 (INS018).

In some embodiments, the insulin-Fc fusion polypeptide comprises a sequence as shown in SEQ ID NO: 77.

In some embodiments, the insulin-Fc fusion polypeptide comprises a sequence as shown in SEQ ID NO: 78.

Further provided herein is an insulin-Fc conjugate comprising at least two insulin-Fc fusion polypeptides provided herein, wherein each of the insulin-Fc fusion polypeptides comprises from N- to C-terminus: an insulin, such as a single chain insulin provided herein, and an Fc region polypeptide.

Typically, the conjugate is a homodimer of two insulin-Fc fusion polypeptides provided herein. Thus, both fusion polypeptides, typically, have the same sequence, i.e. they are identical.

In an embodiment of the provided insulin-Fc conjugate, said insulin is an insulin analog having reduced binding affinity to the human insulin receptor as compared to human insulin. Typically, the insulin is an insulin provided herein (see e.g. Table B), such as the single chain insulin provided herein.

Typically, the conjugate is a conjugate of two insulin-Fc fusion polypeptides, wherein the two insulin-Fc fusion polypeptides are linked by at least two covalent bonds at the C-terminus of the fusion polypeptides. Thus, the two fusion polypeptides are linked by at least two covalent bonds which are located C-terminally to the Fc region polypeptide.

In an embodiment, at least two covalent bonds are located C-terminally to a CH2, CH3 and CH4 domain on each fusion polypeptide.

In an alternative embodiment, at least two covalent bonds are located C-terminally to a CH2 and CH3 domain on each fusion polypeptide.

Typically, the covalent bonds are disulfide bonds.

Typically, said two insulin-Fc fusion polypeptides are not covalently linked by one or more disulfide bonds between the N-terminus of each Fc region polypeptide.

Typically, each the first insulin-Fc fusion polypeptide and the second insulin Fc fusion polypeptide comprise at the C-terminus a C-terminal peptide, wherein said C-terminal peptide comprises at least two cysteine residues allowing the formation of two disulfide bonds between the first and second fusion polypeptide. Typically, said C-terminal peptide comprises the amino acid sequence of an antibody hinge region (or a highly similar sequence).

### Further Aspects

Further provided is a polynucleotide encoding for the single-chain insulin provided herein, or the insulin-Fc fusion polypeptide provided herein.

Further provided is a host cell comprising the insulin-Fc conjugate provided herein, the single-chain insulin provided herein, or the insulin-Fc fusion polypeptide provided, and/or the polynucleotide provided herein.

Further provided is a method of producing the insulin-Fc conjugate provided herein, the single-chain insulin provided herein, or the insulin-Fc fusion polypeptide provided herein, comprising incubating the host cell provided herein under conditions that allow for expressing the single-chain insulin, or the insulin-Fc fusion polypeptide. Expression of the insulin-Fc fusion polypeptide may lead to the formation of the insulin-Fc conjugate as defined herein.

Further provided is a pharmaceutical composition comprising in a pharmaceutically effective amount insulin-Fc conjugate provided herein, the single-chain insulin provided herein, or the insulin-Fc fusion polypeptide provided herein.

Further provided is the insulin-Fc conjugate of provided herein, the insulin provided herein (such as the single chain insulin provided herein), or the insulin-Fc fusion polypeptide provided herein for use as a medicament.

Further provided is the insulin-Fc conjugate of provided herein, the insulin provided herein (such as the single chain insulin provided herein), or the insulin-Fc fusion polypeptide provided herein for use as a medicament for treatment of a disease selected from the group consisting of gestational diabetes, diabetes mellitus type 1, diabetes mellitus type 2, and hyperglycemia and/or for lowering blood glucose levels.

### DESCRIPTION OF THE FIGURES

FIG. 1 Analysis of pharmacodynamic activity (blood glucose lowering activity) in normoglycemic rats for INS007 (SEQ ID NO: 8). Blood glucose lowering activity was observed for at least 168 hrs.

FIG. 2 Analysis of pharmacodynamic activity (blood glucose lowering activity) in normoglycemic rats of INS009 (SEQ ID NO: 10) and INS013 (SEQ ID NO: 14) in normoglycemic rats. Blood glucose lowering activity was observed at least for 144 hrs (INS013 SEQ, ID NO: 14) and at least for 168 hrs (INS009, SEQ ID NO: 10).

FIG. 3 Analysis of pharmacodynamic activity (blood glucose lowering activity) in normoglycemic rats of INS011 (SEQ ID NO: 12), INS018 (SEQ ID NO: 19), and INS019 (SEQ ID NO: 20) in normoglycemic rats. Blood glucose lowering activity was observed at least for 144 hrs (INS018 [SEQ ID NO: 19], INS 019 [SEQ ID NO: 20]) and at least for 168 hrs (INS011, SEQ ID NO: 12).

FIG. 4 Analysis of pharmacodynamic activity (blood glucose lowering activity) in normoglycemic Goettingen minipics of INS007 (SEQ ID NO: 08), INS009 (SEQ ID NO: 10), and INS013 (SEQ ID NO: 14) in healthy female Göttingen Minipigs. Blood glucose lowering activity was observed at least for 216 hrs for all variants. The single-chain-insulin of variant INS009 (SEQ ID NO: 10) is the same as in INS024 (SEQ ID NO: 25) (see FIG. 7). The difference between these two variants is the architecture of the Fc-domain. In INS009 (SEQ ID NO: 10) the single-chain-insulin (SEQ ID NO: 50) is fused to a Fc-domain lacking the hinge at the N-terminus (SEQ ID NO: 34) containing a linker sequence (SEQ ID NO: 42) and a hinge sequence (SEQ ID NO: 41) at the C-terminus.

FIG. 5 Analysis of pharmacodynamic activity (blood glucose lowering activity) in normoglycemic Goettingen minipigs of INS011 (SEQ ID NO: 12). Blood glucose lowering activity was observed at least for 264 hrs.

FIG. 6 Analysis of pharmacodynamic activity (blood glucose lowering activity) in normoglycemic Goettingen minipigs. of INS019 (SEQ ID NO: 20) and INS020 (SEQ ID NO: 21). Blood glucose lowering activity was observed at least for 192 hrs for both variants.

FIG. 7 Analysis of pharmacodynamic activity (blood glucose lowering activity) in normoglycemic Goettingen minipigs. This single-chain-insulin variant is the same single-chain-insulin variant as in INS009 (SEQ ID NO: 10) (see FIG. 4). The only difference is the structure of the used Fc-domain. In INS024 (SEQ ID NO: 25) the single-chain-insulin (SEQ ID NO: 50) is fused via a linker sequence (SEQ ID NO: 29) to the hinge domain of an IgG1-Fc-domain (SEQ ID NO: 37). Blood lowering activity was observed for at least 120 hrs.

FIG. 8 Schematic drawings of insulin-Fc fusion proteins provided herein. A) The insulin B-chain is connected with a linker 1 to the insulin A-chain. This single-chain-insulin is connected via a linker 2 to the IgG Fc-domain lacking the hinge region. The hinge region is relocated to the C-terminus of the Fc-domain. The two Fc-chains are connected via disulfide bridges in the C-terminal hinge. B) Schematic structure of a single-chain-insulin-fused to the hinge region of a IgG. The two Fc-protein chains are connected via disulfide bridges in the N-terminal hinge.

FIG. 9 Time course of blood glucose lowering of INS011 (SEQ ID NO: 10) and INS026 (SEQ ID NO: 78) in normoglycemic rats. Blood glucose lowering activity was observed at least for 180 hrs (INS011) and for at least 168 hrs (INS026). In both constructs, linker sequence to the Fc-domain (SEQ ID NO: 29) and Fc-domain are the same (SEQ ID NO: 34) with C-terminal “hinge” sequences SEQ ID NO: 42 and SEQ ID 41.

FIG. 10 Time course of blood glucose lowering of INS007 (SEQ ID NO: 6) and INS027 (SEQ ID NO: 79) in normoglycemic rats. Blood glucose lowering activity was observed at least for 168 for both variants.

FIG. 11 Mean+SD plasma concentration of INS009 (SEQ ID NO: 10), INS011 (SEQ ID NO: 12) and INS013 (SEQ ID NO: 14) after the administration of 1 nmol/kg subcutanuos single dose to male cynomolgus monkey.

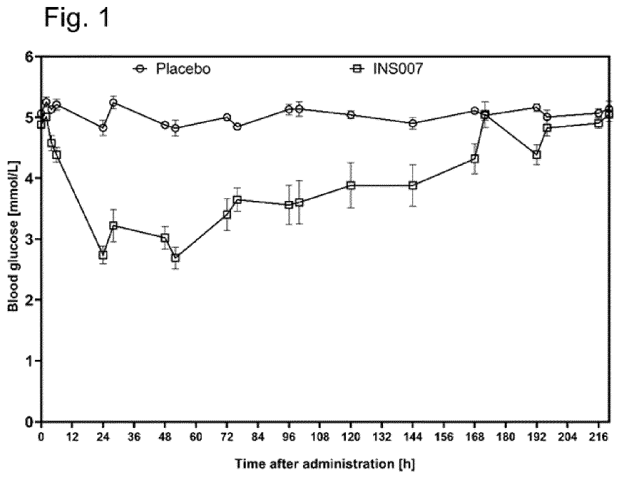

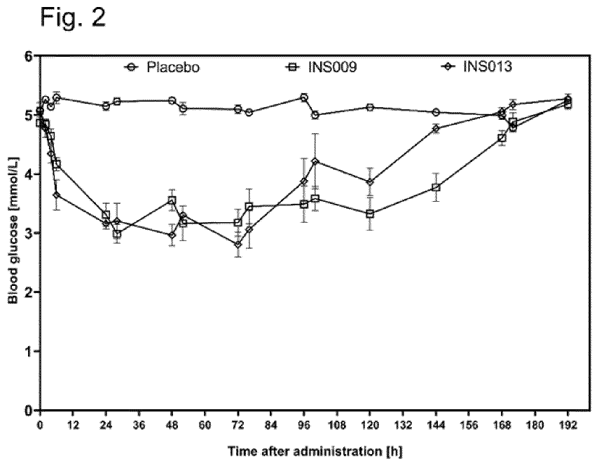

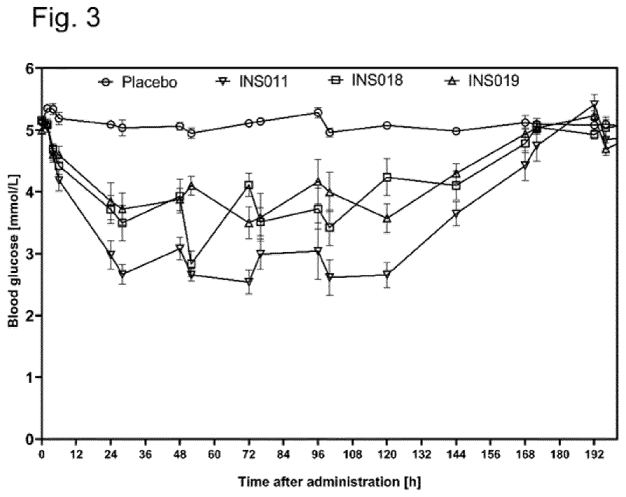

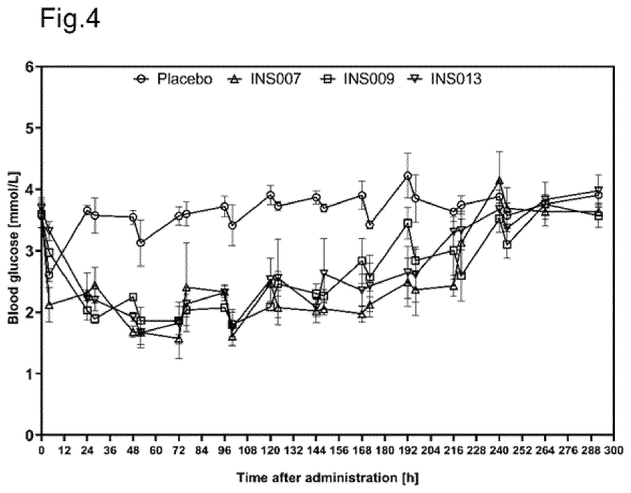

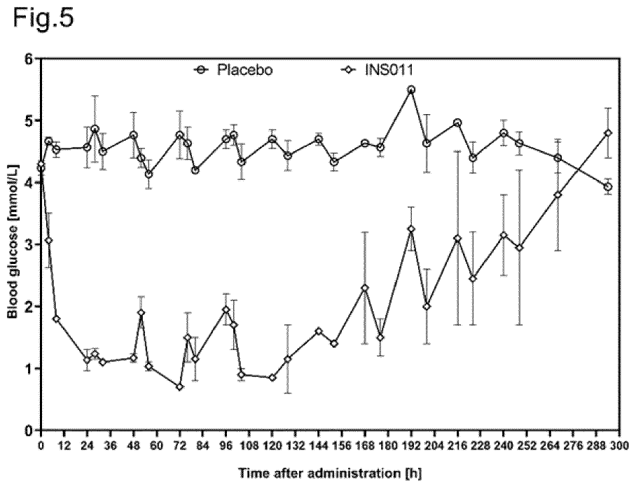

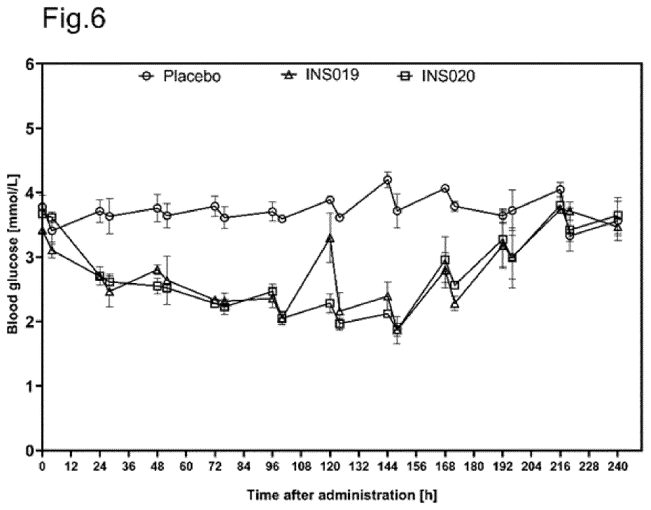

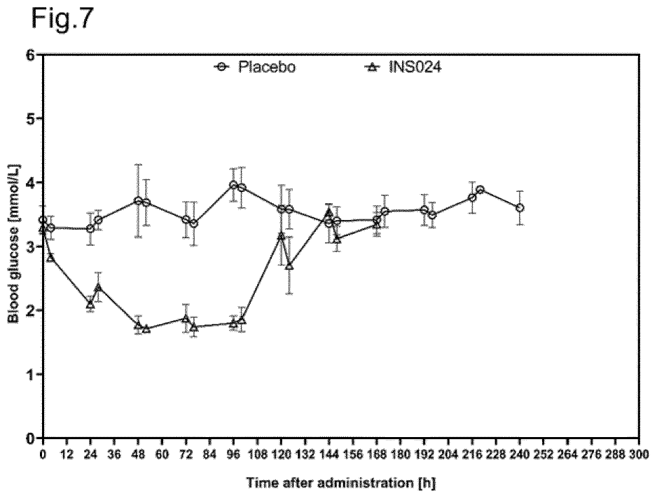

FIG. 12 Sequence of single-chain-insulin-Fc fusion polypeptide INS009 (SEQ ID NO: 10). The fusion polypeptide comprises from N-to-C terminus:
a) an insulin B chain (SFVNQHLCGSHLVEALELVCGERGFHYTPKT (SEQ ID NO: 73),
b) a first linker peptide (GSYPGGV, SEQ ID NO: 26),
c) an insulin A chain (GIVEQCCTSICSLEQLENYCG, SEQ ID NO: 74),
d) a second linker (GEAEDLQVGQVELGGGPGAGSLQPEGSLQ, SEQ ID NO: 29),
e) an IgG region without a hinge at the normal position (italics, SEQ ID NO: 34),
f) a third linker peptide (GGGGSA, SEQ ID NO: 42),
g) a C-terminal peptide, the amino acid sequence of an antibody hinge region comprising two cysteine residues (SEQ ID NO: 41, ESKYGPPCPPCPA).

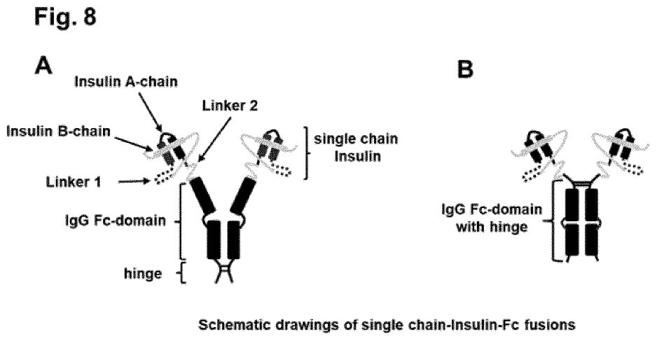

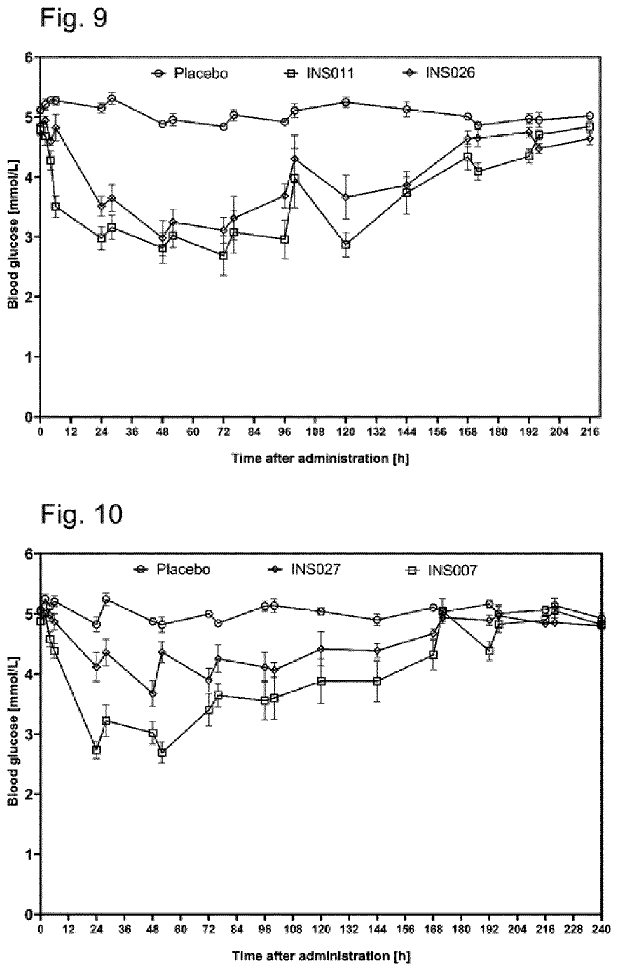

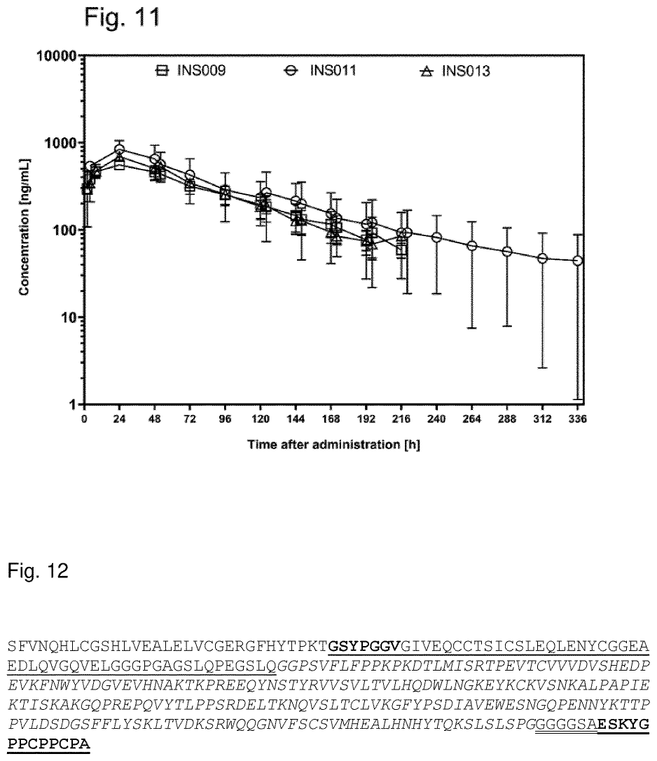

Two single-chain-insulin-Fc fusion polypeptides form an insulin Fc conjugate via formation of two disulfide bonds between two cysteine residues present in the C-terminal hinge region, see FIG. 8A).

### Definitions

As used in this description and the appended claims, the singular forms “a”, “an”, and “the” include plural referents, unless the content clearly dictates otherwise.

As used herein, the term “comprising” is to be interpreted as specifying the presence of the stated features, integers, steps or components as referred to, but does not preclude the presence or addition of one or more features, integers, steps or components, or groups thereof. Thus, e.g., a protein comprising a sequence of amino acids, may comprise more amino acids than the actually cited ones, i.e., be embedded in a larger protein.

A “polypeptide”, as used herein, is a chain of 10 or more amino acids which are linked covalently by peptide bonds. Accordingly, the term “polypeptide” can stand for a chain of a multi-chain protein, independent of the length of such chain. In some embodiments, a polypeptide is a chain of a multi-chain protein.

A “linker” or “linker peptide”, as used herein, is a short and flexible amino acid sequence which links two regions of a molecule. For instance, a linker can link the B-chain of an insulin with the A-chain. This linker is referred to herein as “first linker peptide”. Moreover, a linker can link a single-chain insulin with the Fc region. This linker is referred to herein as “second linker peptide”. Further, a linker peptide can link the Fc region and the C-terminal region of the molecule which comprises the at least two inter-chain covalent bonds. This linker is referred to herein as “third linker peptide”

The expression “insulin analog” as used herein refers to a peptide which has a molecular structure which formally can be derived from the structure of a naturally occurring insulin (herein also referred to as “parent insulin”, e.g. human insulin).

The expression “parent insulin” as used herein refers to naturally occurring insulin, i.e. to an unmutated, wild-type insulin. In some embodiments, the parent insulin is animal insulin, such as mammalian insulin. For example, the parent insulin may be human insulin, porcine insulin, or bovine insulin. Typically, the parent insulin is human insulin.

The B-chain of human insulin comprises the following sequence:

(SEQ ID NO: 32)
FVNQHLCGSHLVEALYLVCGERGFFYTPKT

The A-chain of human insulin comprises the following sequence:

(SEQ ID NO: 33)
GIVEQCCTSICSLYQLENYCN

In an embodiment, the insulin analog provided herein or the insulin analog comprised by the insulin-Fc fusion polypeptide provided herein is a single-chain insulin. A single-chain insulin is a single polypeptide chains in which the insulin B-chain is linked contiguously with the insulin A-chain via a connecting peptide, herein referred to as first linker peptide.

The insulin analog provided herein or the insulin analog comprised by the insulin-Fc fusion polypeptide provided herein provided herein comprise at least one mutation (substitution, deletion, or addition of an amino acid) relative to parent insulin, typically. The term “at least one”, as used herein means one, or more than one, such as “at least two”, “at least three”, “at least four”, “at least five”, etc. In some embodiments, the insulin analogs provided herein comprise at least one mutation in the B-chain and at least one mutation in the A-chain. In a further embodiment, the insulin analogs provided herein comprise at least two mutations in the B-chain and at least one mutation in the A-chain.

Typically, the insulin analogs provided herein or the insulin analogs comprised by the insulin-Fc fusion polypeptide provided herein provided herein comprises two peptide chains, an A-chain and a B-chain. Typically, the two chains are connected by disulfide bridges between cysteine residues. For example, in at least one embodiment, the human insulin analog comprises three disulfide bridges: one disulfide bridge between the cysteines at position A6 and A11, one disulfide bridge between the cysteine at position A7 of the A-chain and the cysteine at position B7 of the B-chain, and one between the cysteine at position A20 of the A-chain and the cysteine at position B19 of the B-chain.

Typically, the insulin analogs provided herein or the insulin analogs comprised by the insulin-Fc fusion polypeptide provided herein have an insulin receptor binding affinity which is reduced as compared to the insulin receptor binding affinity of the corresponding parent insulin, e.g. of human insulin. Thus, the insulin analogs have a very low clearance rate, i.e. a very low insulin-receptor-mediated clearance rate.

The insulin receptor can be any mammalian insulin receptor, such as a bovine, porcine or human insulin receptor. In some embodiments, the insulin receptor is a human insulin receptor, e.g. human insulin receptor isoform A or human insulin receptor isoform B.

Typically, the insulin analog provided herein or the insulin analog comprised by the insulin-Fc fusion polypeptide provided herein have, i.e. exhibit, less than 25% of the binding affinity to the human insulin receptor isoform B compared to human insulin, such as less than 15%. In an embodiment, the insulin analog has 10 to 25% of the binding affinity to the human insulin receptor isoform B compared to human insulin.

Typically, the insulin analog provided herein or the insulin analog comprised by the insulin-Fc fusion polypeptide provided herein have, i.e. exhibit, less than 15% of the binding affinity to the human insulin receptor isoform a compared to human insulin, such as less than 8%. In an embodiment, the insulin analog has 1 to 15%, such as 3% to 10% of the binding affinity to the human insulin receptor isoform A compared to human insulin.

Methods for determining the binding affinity of an insulin analog to an insulin receptor are well known in the art. For example, the insulin receptor binding affinity can be determined by a scintillation proximity assay which is based on the assessment of competitive binding between [125I]-labelled parent insulin, such as [125I]-labelled human insulin, and the (unlabeled) insulin analog to the insulin receptor. The insulin receptor can be present in a membrane of a cell, e.g. of CHO (Chinese Hamster Ovary) cell, which overexpresses a recombinant insulin receptor. In an embodiment, the insulin receptor binding affinity is determined as described in the Examples section of WO2020/120463 A1 which herewith is incorporated by reference with respect to the entire disclosure content.

Binding of a naturally occurring insulin or an insulin analog to the insulin receptor activates the insulin signaling pathway. The insulin receptor has tyrosine kinase activity. Binding of insulin to its receptor induces a conformational change that stimulates the autophosphorylation of the receptor on tyrosine residues. The autophosphorylation of the insulin receptor stimulates the receptor's tyrosine kinase activity toward intracellular substrates involved in the transduction of the signal. The autophosphorylation of the insulin receptor by an insulin analog is therefore considered as a measure for signal transduction caused by said analog. In some embodiments, the insulin analog provided herein are capable of inducing 10 to 25% autophosphorylation of human insulin receptor isoform B relative to human insulin and/or 1 to 15% autophosphorylation of human insulin receptor isoform A relative to human insulin. Further, in some embodiments, the insulin analogs provided herein are capable of inducing 3 to 7%, such as 5 to 7% insulin receptor autophosphorylation relative to the parent insulin (such as human insulin). The insulin receptor autophosphorylation relative to a parent insulin can be determined as described in the Examples section.

In the insulin-Fc fusion polypeptide provide herein, an insulin, such as a single-chain insulin, shall be operably linked to an Fc region polypeptide. In antibodies, the Fc (fragment crystallizable) region is the region that interacts with cell surface receptors called Fc receptors, thereby activating the immune system. The Fc regions of IgGs are known to bear a highly conserved N-glycosylation site which is essential for Fc receptor-mediated activity.

The two insulin-Fc fusion polypeptides present in the conjugate provided herein form an antibody Fc region.

An “Fc region”, as used herein, is a fragment of an immunoglobulin molecule which is formed by several constant heavy chain (CH) immunoglobulin domains of two polypeptides. In natural antibodies, the Fc region mediates binding to Fc receptors and components of the complement system. For IgG, IgA and IgD, the Fc region is formed by the CH2 and CH3 constant domains of both heavy chains. For IgM and IgE, the Fc region is formed by the CH2, CH3 and CH4 constant domains of both heavy chains. The Fc regions of the present invention can be formed by the same CH domains as the Fc region of natural antibodies, i.e. CH2 and CH3 domains or CH2, CH3 and CH4 domains. The Fc regions of the present invention comprise CH domains which are either identical to CH domains of the natural immunoglobulins or are derived from CH domains of the natural immunoglobulins, e.g. by inserting one or several points mutations.

The term “Fc region polypeptide” is a fragment of an antibody heavy chain, said fragment comprising the constant domains of the antibody heavy chain. Thus, the Fc region polypeptide, typically, comprises the constant domain of an IgG, IgM, IgA, IgD or IgE antibody heavy chain. For example, if the antibody is an IgG antibody, the Fc region polypeptide comprises the CH2 and CH3 domains (of the antibody heavy chain). In an embodiment, the Fc region polypeptide comprises or consists of a) an amino acid sequence as shown in SEQ ID NO: 34, orb) a sequence being at least 90%, at least 91%, at least 92%, at least 93%, at least 94%, at least 95%, at least 96%, at least 97%, at least 98% or at least 99% identical to SEQ ID NO: 34. Typically, the two Fc region polypeptide form an antibody Fc region in the conjugate provided herein.

Said Fc region polypeptide may comprise additional elements such as a third linker peptide a C-terminal peptide allowing the formation of said at least two covalent bonds between said two insulin-Fc fusion polypeptides. Typically, however, said Fc region polypeptide lacks an antibody hinge region at the N-terminus.

The term “hinge region”, as used herein, relates to a part of an antibody sequence which is located between the Fc region and the Fab regions. It provides segmental flexibility and can stabilize the antibody molecules. The hinge region can be clearly defined based on structural data, e.g. the IgG1 hinge region comprises the residues 221-237 (R. Nezlin, “The Immunoglobulins”, Academic Press, 1998, pages 23-26). In IgG antibodies, the hinge region comprises disulfide bonds which link the two heavy chains of the antibody. IgG1 and IgG4 comprise 2 such disulfide bonds, while IgG2 comprises 4 and IgG3 comprises 11 (Liu and May 2012 MAbs. 2012 January-February; 4(1): 17-23).

Herein, a region is typically only designated as “hinge region” if it is located N-terminally of the Fc region, as it is the case for all natural antibodies. If an amino acid sequence which is identical or highly similar to a hinge region is placed C-terminally of the Fc region (as in some embodiments of molecules of the present invention), it is designated as “amino acid sequence comprising the amino acid sequence of an (antibody) hinge region” or the like. Highly similar means at least 70% identity to the sequence of an antibody hinge region and/or comprising at least 7 contiguous amino acids of the sequence of an antibody hinge region.

A “covalent bond”, as used herein, is a chemical bond that involves the sharing of electron pairs between atoms. A covalent bond is distinct from non-covalent interaction, such as electrostatic interactions or hydrophobic effect.

The indication that a covalent bond, e.g. disulfide bonds, is “located C-terminally” of an Fc region means that, on both polypeptides forming said Fc region, the amino acid residues, e.g. cysteine residues, which are involved in the formation of said covalent bond, are located C-terminally of the portion of the polypeptides which form the Fc region.

The indication that no covalent bond, e.g. disulfide bond, is “located N-terminally” of an Fc region means that, on both polypeptides forming said Fc region, there are no amino acid residues, e.g. cysteine residues, which are involved in the formation of such a covalent bond, located N-terminally of the portion of the polypeptide which form the Fc region.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity. Typically, standard parameters are applied for determining the degree of sequence identity of two sequences. In some embodiments, the degree of sequence identity is calculated over the whole length of the two sequences. In some embodiments, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85:2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are typically employed to determine their optimal alignment and, thus, the degree of identity. Typically, the default values of 5.00 for gap weight and 0.30 for gap weight length are used. In an embodiment, the percent identity between two amino acid sequences is determined using the Needleman and Wunsch algorithm (Needleman 1970, J. Mol. Biol. (48):444-453) which has been incorporated into the needle program in the EMBOSS software package (EMBOSS: The European Molecular Biology Open Software Suite, Rice, P., Longden, I., and Bleasby, A., Trends in Genetics 16(6), 276-277, 2000), a BLOSUM62 scoring matrix, and a gap opening penalty of 10 and a gap extension penalty of 0.5. A preferred, non-limiting example of parameters to be used for aligning two amino acid sequences using the needle program are the default parameters, including the EBLOSUM62 scoring matrix, a gap opening penalty of 10 and a gap extension penalty of 0.5.

The term “at least 80% identical” with respect to two sequences means that the two sequences have 80% sequence identity or more. In some embodiments, the sequences are at least 85% or 87% identical. In some embodiments, the sequences are at least 90% identical. In some embodiments, the sequences are at least 91%, at least 92%, at least 93%, at least 94%, at least 95%, at least 96%, at least 97%, at least 98%, or at least 99% identical. In some embodiments, the sequences are identical, i.e. 100% identical.

The term “cell” or “host cell”, as used herein, refers to an intact cell, i.e., a cell with an intact membrane that has not released its normal intracellular components such as enzymes, organelles, or genetic material. In certain exemplary embodiments, an intact cell is a viable cell, i.e., a living cell capable of carrying out its normal metabolic functions. In certain exemplary embodiments, a cell or a host cell is any cell which can be transfected or transformed with an exogenous nucleic acid. In certain exemplary embodiments, the cell, when transfected or transformed with an exogenous nucleic acid and transferred to a recipient, can express the nucleic acid in the recipient.

The term “cell” includes prokaryotic cells, such as bacterial cells, and eukaryotic cells, such as yeast cells, fungal cells or mammalian cells. Suitable bacterial cells include, but are not limited to, cells from gram-negative bacterial strains, such as strains of Escherichia coli, Proteus, and Pseudomonas, and gram-positive bacterial strains, such as strains of Bacillus, Streptomyces, Staphylococcus, and Lactococcus. Suitable fungal cells include, but are not limited to, cells from the species of Trichoderma, Neurospora, and Aspergillus. Suitable yeast cells include, but are not limited to, cells from the species of Saccharomyces (for example, Saccharomyces cerevisiae), Schizosaccharomyces (for example, Schizosaccharomyces pombe), Pichia (for example, Pichia pastoris and Pichia methanolica), and Hansenula. Suitable mammalian cells include, but are not limited to, for example, CHO (Chinese Hamster Ovary) cells, BHK cells, Hela cells, COS cells, HEK-293 and the like. In one embodiment, HEK-293 cells are used. In another embodiment, CHO cells are used. However, amphibian cells, insect cells, plant cells, and any other cells used in the art for the expression of heterologous proteins can be used as well. In certain exemplary embodiments, mammalian cells (e.g., cells from humans, mice, hamsters, pigs, goats, or primates) are used for adoptive transfer.

In some embodiments, the host cell comprises the polynucleotide encoding the insulin, fusion protein or insulin-Fc conjugate provided herein, and/or vector comprising said polynucleotide. Typically, said vector is an expression vector.

A cell or host cell may be isolated or part of a tissue or organism, such as a “non-human organism”. The term “non-human organism”, as used herein, is meant to include non-human primates or other animals, e.g., mammals, such as cows, horses, pigs, sheep, goats, dogs, cats, rabbits or rodents (e.g., mice, rats, guinea pigs and hamsters). In some embodiment, the non-human organism is a cynomolgus monkey.

A pharmaceutical composition as set forth herein typically comprises the insulin or insulin-Fc conjugate provided herein together with a pharmaceutically acceptable carrier and/or a pharmaceutically acceptable excipient. The term “pharmaceutically acceptable”, as used herein, refers to the non-toxicity of a material which, in certain exemplary embodiments, does not interact with the action of the active agent of the pharmaceutical composition, i.e. the insulin or insulin-Fc conjugate.

The term “carrier”, as used herein, refers to an organic or inorganic component, of a natural or synthetic nature, in which the active component is combined in order to facilitate, enhance or enable application. Typically, the term “carrier” also includes one or more compatible solid or liquid fillers, diluents or encapsulating substances, which are suitable for administration to a subject.

Suitable carrier substances for parenteral administration include, but are not limited to, sterile water, Ringer's solution, Lactated Ringer's solution, physiological saline, bacteriostatic saline (e.g., saline containing 0.9% benzyl alcohol), phosphate-buffered saline (PBS), Hank's solution, polyalkylene glycols, hydrogenated naphthalenes and, in particular, biocompatible lactide polymers, lactide/glycolide copolymers or polyoxyethylene/polyoxy-propylene copolymers.

The term “excipient”, as used herein, is intended to include all substances which may be present in a pharmaceutical composition and which are not active ingredients, such as salts, binders (e.g., lactose, dextrose, sucrose, trehalose, sorbitol, mannitol), fillers, lubricants, thickeners, surface active agents, preservatives, emulsifiers, buffer substances, flavoring agents, or colorants.

The form of the pharmaceutical composition, the route of administration, the dosage and the regimen naturally depend upon the condition to be treated, the severity of the illness, the age, weight, and gender of the patient, etc.

The pharmaceutical composition can be formulated for a topical, oral, parenteral, intranasal, intravenous, intramuscular, subcutaneous or intraocular administration and the like. In some embodiments, the composition is formulated for intravenous administration. In some embodiments, the composition is formulated for subcutaneous administration.

In some embodiments, the pharmaceutical compositions contain vehicles which are pharmaceutically acceptable for a formulation capable of being administered parenterally, such as intravenously or subcutaneously. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The insulin or insulin-Fc conjugate described herein may be administered via any conventional route, e.g., orally, pulmonary administration, by inhalation or parenterally, including by injection or infusion. In some embodiments, parenteral administration is used, such as intravenous, intraarterial, subcutaneous, intradermal or intramuscular administration. In some embodiments, the insulin or insulin-Fc conjugate provided herein is administered subcutaneously.

The insulin or insulin-Fc conjugate, or the pharmaceutical composition described herein are usually administered in therapeutically effective amounts. The term “therapeutically effective amount” is understood by the skilled person. In some embodiments, the term refers to an amount which achieves a desired therapeutic reaction or a desired therapeutic effect alone or together with further doses, optionally without causing or only minimally causing unacceptable or unwanted side-effects.

Typically, the activity of an insulin analog is provided in terms of units of human insulin, which is the current standard of insulin dosing for patients. The World Health Organization currently defines one unit of human insulin (EP/US/IU) as 0.0347 mg of human insulin human (Burns C, Morris T, Jones B, et al. World Health Organization. Proposal to Initiate a Project to Evaluate a Candidate International Standard for Human Recombinant Insulin. WHO/BS/10.2143-Working document QAS/10.381, 2010). One unit of an insulin analog, typically, is biologically equivalent to one unit of human insulin.

In an embodiment, the insulin analog provided herein or the insulin-Fc conjugate provided herein is administered in an amount which corresponds to 200 to 1500 U of human insulin, such as 400 U to 1000 U, such as 400 U, 450 U, 500, U, 600 U, 700 U, 800 U, 900 U, or 1000 U.

In an embodiment, the insulin-Fc conjugate provided herein is administered once weekly.

For example, the insulin analog provided herein or the insulin-Fc conjugate provided herein is administered once weekly in an amount of 0.01-100 U/kg body weight, such as 1 to 20 U/kg body weight, such as 1 to 10 U/kg body weight.

The terms “subject” and “patient” are used interchangeably herein. The “subject” or “patient” may be a vertebrate. The term includes both humans and other animals, particularly mammals, and other organisms. Accordingly, herein the subject may be an animal such as a mouse, rat, hamster, rabbit, guinea pig, ferret, cat, dog, chicken, sheep, bovine species, horse, camel, or primate. In some embodiments, the subject is a mammal. In some embodiments, the subject is a primate. In some embodiments, the subject is human. In some embodiments, the subject is 16 years old, or older.

In some embodiments, the subject is suffering from a disease or disorder as referred to herein. For example, the subject may be an obese subject. In some embodiments, the patient is at risk of suffering from a disease or disorder as referred to herein.

Typically, the term “disease or disorder” refers to any pathological or unhealthy state which can be treated by administering the insulin or insulin-Fc conjugate, or the pharmaceutical composition provided herein, in particular diabetes mellitus (such as gestational diabetes, diabetes mellitus type 1 or diabetes mellitus type 2) and or hyperglycemia.

“Diabetes mellitus” (also simply referred to as “diabetes”), as used herein, refers to a group of metabolic diseases characterized by high levels of blood glucose resulting from defects in insulin production, insulin action, or both. In one embodiment, diabetes mellitus is selected from the group consisting of type 1 diabetes mellitus, type 2 diabetes mellitus, gestational diabetes mellitus. The current WHO diagnostic criteria for diabetes mellitus are as follows: fasting plasma glucose≥7.0 mmol/l (126 mg/dL) or 2-hour plasma glucose≥11.1 mmol/l (200 mg/dL).

In some embodiments, diabetes is type 1 diabetes mellitus. “Type 1 diabetes mellitus” as used herein, is a condition characterized by high blood glucose levels caused by total lack of insulin. This occurs when the body's immune system attacks the insulin producing beta cells in the pancreas and destroys them. The pancreas then produces little or no insulin. Pancreatic removal or disease may also lead to loss of insulin-producing beta cells. Type 1 diabetes mellitus accounts for between 5% and 10% of cases of diabetes.

In some embodiments, diabetes is type 2 diabetes mellitus. “Type 2 diabetes mellitus” as used herein, is a condition characterized by excess glucose production in spite of the availability of insulin, and circulating glucose levels remain excessively high as a result of inadequate glucose clearance (insulin action).

In some embodiments, diabetes is gestational diabetes. “Gestational diabetes”, as used herein, is a condition in which women without previously diagnosed diabetes exhibit high blood glucose levels during pregnancy (especially during the third trimester). Gestational diabetes affects 3-10% of pregnancies, depending on the population studied.

The term “hyperglycemia”, as used herein, refers to an excess of sugar (glucose) in the blood.

The term “treating” or “treatment”, as used herein, refers to the administration of a compound or composition or a combination of compounds or compositions to a subject in order to: prevent, ameliorate, or eliminate a disease and/or disorder as referred to herein, such as diabetes mellitus, in a subject. Thus, the term encompasses both the treatment of an existing disease or disorder as referred to herein, or prevention of disease or disorder, i.e. prophylaxis. It will therefore be recognized that treatment as referred to herein may, in some embodiments, be prophylactic. In some embodiments, the term refers to the treatment of an existing disease or disorder as referred to herein. Thus, the subject is suffering from said disease or disorder.

### DETAILED DESCRIPTION OF THE PRESENT INVENTION

### First Aspect: Insulins

Provided herein are insulin analogs comprising an insulin B chain and the corresponding insulin A chain as shown Table B in the Examples section. The insulin analogs are typically provided as single chain insulins.

Provided herein is an insulin analog comprising an insulin B chain and an insulin A chain, wherein the insulin B chain comprises or consists of the amino acid sequence SFVNQHLCGSHLVEALELVCGERGFHYTPKT (SEQ ID NO: 69) and the insulin A chain comprises or consists of the amino acid sequence GIVEQCCTSICSLEQLENYCG (SEQ ID NO: 74).

Further provided is an insulin analog comprising an insulin B chain and an insulin A chain, wherein the insulin B chain comprises or consists of the amino acid sequence SFVNQHLCGSHLVEALHLVCGERGFAYTPKT (SEQ ID NO: 70) and the insulin A chain comprises or consists of the amino acid sequence GIVEQCCTSICSLEQLENYCG (SEQ ID NO: 74).

Further provided is an insulin analog comprising an insulin B chain and an insulin A chain the insulin B chain wherein the insulin B chain comprises or consists of the amino acid sequence FVNQHLCGSHLVEALHLVCGERGFHYTPKT (SEQ ID NO: 71) and the insulin A chain comprises or consists of the amino acid sequence GIVEQCCTSICSLSQLEDYCG (SEQ ID NO: 75).

Further provided is an insulin analog comprising from an insulin B chain and an insulin A chain the insulin B chain comprises or consists of the amino acid sequence SFVNQHLCGSHLVEALELVCGERGFHYTPKT (SEQ ID NO: 73) and the insulin A chain comprises or consists of the amino acid sequence GIVEQCCTSICSLSQLEDYCG (SEQ ID NO: 75).

Further provided is an insulin analog comprising an insulin B chain and an insulin A chain, wherein the insulin B chain comprises or consists of the amino acid sequence the insulin B chain comprises or consists of the amino acid sequence SFVNQHLCGSHLVEALYLVCGERGFFYTPKT (SEQ ID NO: 65) and wherein the insulin A chain comprises or consists of the amino acid sequence GIVEQCCTSICSLEQLENYCG (SEQ ID NO: 74).

Further provided is an insulin analog comprising an insulin B chain and an insulin A chain the insulin B chain, wherein the insulin B chain comprises or consists of the amino acid sequence SFVNQHLCGSHLVEALHLVCGERGFAYTDKT (SEQ ID NO: 66) and wherein the insulin A chain comprises or consists of the amino acid sequence GIVEQCCTSICSLEQLENYCG (SEQ ID NO: 74).

Further provided is an insulin analog comprising an insulin B chain and an insulin A chain the insulin B chain, wherein the insulin B chain comprises or consists of the amino acid sequence SFVNQHLCGSHLVEALHLVCGERGFAYTDKT (SEQ ID NO: 67) and wherein the insulin A chain comprises or consists of the amino acid sequence GIVEQCCTSICSLEQLENYCG (SEQ ID NO: 74).

Typically, the insulin analog provided herein is provided as single chain insulin. In the single chain insulin, the insulin B chain is, typically, linked to the insulin A chain via a first linker peptide.

Accordingly, said single chain insulin comprises from N- to C-terminus:
a) the insulin B chain,
b) a first linker peptide, and
c) the insulin A chain.

In an embodiment, the first linker peptide has a length of 1 to 50 amino acids. In another embodiment, the first linker peptide has a length of 5 to 15 amino acids. In another embodiment, the first linker peptide has a length of 5 to 10 amino acids. In another embodiment, the first linker peptide has a length of 7 amino acids.

Typically, the first linker peptide comprises or consists of the amino acid sequence EEYPGDV (SEQ ID NO: 27). Also typically, the first linker peptide comprises or consists of the amino acid sequence GSYPGGV (SEQ ID NO: 26).

In an embodiment, the single chain insulin comprises or consists of the following amino acid sequence:

(SEQ ID NO: 50)
SFVNQHLCGSHLVEALELVCGERGFHYTPKTGSYPGGVGIVEQCCTSICSLEQLENYCG.

In another embodiment, the single chain insulin comprises or consists of the following amino acid sequence:

(SEQ ID NO: 52)
SFVNQHLCGSHLVEALHLVCGERGFAYTPKTGSYPGGVGIVEQCCTSICSLEQLENYCG.

In another embodiment, the single chain insulin comprises or consists of the following amino acid sequence:

(SEQ ID NO: 53)
FVNQHLCGSHLVEALHLVCGERGFHYTPKTGSYPGGVGIVEQCCTSICSLSQLEDYCG.

In another embodiment, the single chain insulin comprises or consists of the following amino acid sequence:

(SEQ ID NO: 58)
FVNQHLCGSHLVEALELVCGERGFHYTPKTGSYPGGVGIVEQCCTSICSLSQLEDYCG.

In another embodiment, the single chain insulin comprises or consists of the following amino acid sequence:

(SEQ ID NO: 45)
SFVNQHLCGSHLVEALYLVCGERGFFYTPKTEEYPGDVGIVEQCCTSICSLEQLENYCG.

In another embodiment, the single chain insulin comprises or consists of the following amino acid sequence:

(SEQ ID NO: 81)
SFVNQHLCGSHLVEALYLVCGERGFFYTPKTGSYPGGVGIVEQCCTSICSLEQLENYCG.

In another embodiment, the single chain insulin comprises or consists of the following amino acid sequence:

(SEQ ID NO: 82)
SFVNQHLCGSHLVEALHLVCGERGFAYTDKTEEYPGDVGIVEQCCTSICSLEQLENYCG.

In another embodiment, the single chain insulin comprises or consists of the following amino acid sequence:

(SEQ ID NO: 47)
SFVNQHLCGSHLVEALELVCGERGFHYTDKTEEYPGDVGIVEQCCTSICSLEQLENYCG.

### Second Aspect: Insulin Fc Fusions Polypeptides and Conjugates Thereof

Further provided herein are insulin-Fc fusion polypeptides comprising from N- to C-terminus: an insulin and an Fc region polypeptide.

Also provided an insulin-Fc conjugate of two insulin-Fc fusion polypeptides provided herein. Said two insulin-Fc fusion polypeptides shall form an antibody Fc region (via the Fc region)

Typically, the conjugate is a homodimer of two insulin-Fc fusion polypeptides provided herein. Thus, the two insulin-Fc fusion polypeptides, typically, have identical amino acid sequences.

Typically, the insulin comprised by the insulin-Fc fusion polypeptide provided herein is an insulin analog having reduced binding affinity to the human insulin receptor as compared to human insulin.

Typically, the insulin-Fc fusion polypeptide comprises from N- to C-terminus:
a) an insulin B chain,
b) a first linker peptide,
c) an insulin A chain,
d) a second linker peptide,
e) an Fc region (herein also referred to a as Fc region polypeptide).

In an embodiment of the insulin-Fc fusion polypeptide, said insulin is a single-chain insulin, such as any one of the single chain insulins shown in Table B in the Examples section. Typically, said insulin is a single-chain insulin comprising from N- to C-terminus an insulin B chain, a first linker peptide, and an insulin A chain.

In an embodiment of the insulin-Fc fusion polypeptide, the single chain insulin comprises or consists of the following amino acid sequence:

(SEQ ID NO: 50)
SFVNQHLCGSHLVEALELVCGERGFHYTPKTGSYPGGVGIVEQCCTSICSLEQLENYCG,
(SEQ ID NO: 52)
SFVNQHLCGSHLVEALHLVCGERGFAYTPKTGSYPGGVGIVEQCCTSICSLEQLENYCG,
(SEQ ID NO: 53)
FVNQHLCGSHLVEALHLVCGERGFHYTPKTGSYPGGVGIVEQCCTSICSLSQLEDYCG,
(SEQ ID NO: 58)
FVNQHLCGSHLVEALELVCGERGFHYTPKTGSYPGGVGIVEQCCTSICSLSQLEDYCG,
(SEQ ID NO: 45)
SFVNQHLCGSHLVEALYLVCGERGFFYTPKTEEYPGDVGIVEQCCTSICSLEQLENYCG
(SEQ ID NO: 81)
SFVNQHLCGSHLVEALYLVCGERGFFYTPKTGSYPGGVGIVEQCCTSICSLEQLENYCG
(SEQ ID NO: 82)
SFVNQHLCGSHLVEALHLVCGERGFAYTDKTEEYPGDVGIVEQCCTSICSLEQLENYCG,
or
(SEQ ID NO: 47)
SFVNQHLCGSHLVEALELVCGERGFHYTDKTEEYPGDVGIVEQCCTSICSLEQLENYCG

Typically, the single chain insulin is linked to the Fc region polypeptide via a second linker peptide. Said second linker peptide, typically, has a length of 1 to 100, such as of 5 to 40 amino acids. In an embodiment of insulin-Fc fusion polypeptide, the second linker is the human insulin C-peptide, or is a fragment thereof.

The human C-peptide has the following amino acid sequence:

(SEQ ID NO: 31)
EAEDLQVGQVELGGGPGAGSLQPLALEGSLQ.

A fragment of the human C-peptide is, e.g., a fragment having a length of at least 5, 10, 15, 20, or 22 amino acids of the human C-peptide having a sequence as shown in SEQ ID NO: 31.

In an embodiment, the amino acids Leu26Ala27Leu28 (indicated in bold in SEQ ID NO: 31 above) in the C-peptide are deleted in the linker. Thus, said second linker comprises or consists of the amino acid sequence EAEDLQVGQVELGGGPGAGSLQPEGSLQ (SEQ ID NO: 8).

One or more amino acids may be added to the second linker, such as a glycine residue at the N-terminus in order to increase the distance from the second linker to the insulin. Thus, said second linker may comprise or consist of the amino acid sequence GEAEDLQVGQVELGGGPGAGSLQPEGSLQ (SEQ ID NO: 29).

Also, it is envisaged that that the second linker comprises or consists of the amino acid sequence EAEDLQVGQVELGG (SEQ ID NO: 28).

In an embodiment, the Fc region present in the Fc region polypeptide of the present invention is the Fc region of an IgG, IgM, IgA, IgD or IgE antibody. Thus, the Fc region polypeptide, typically comprises the heavy chain constant domains, such as the CH2 and CH3 of an IgG antibody.

In an embodiment, the Fc region is the Fc region of an IgG antibody. Thus, the two polypeptides form an Fc region of an IgG antibody. For example, the Fc region may be the Fc region of an IgG1 antibody. Alternatively, the Fc region may be the Fc region of an IgG4 antibody.

In an embodiment, the Fc region present in the fusion polypeptide provided herein does not comprise, i.e. lacks, an antibody hinge region located N-terminally to the Fc region. Thus, the two fusion polypeptides which form the conjugates are not linked by one or more disulfide bond located N-terminally to the portion of each fusion polypeptide which forms the Fc region.

Typically, the insulin Fc fusion polypeptides are covalently linked by at least two covalent bonds at the C-terminus of the fusion polypeptides. For example, they are covalently linked by two covalent bonds at the C-terminus of the fusion polypeptides.

The expression “at the C-terminus of the fusion polypeptides” encompasses, for example, the 20 C-terminal amino acids of the fusion polypeptide.

In some embodiments, at least two covalent bonds are located C-terminally to a CH2, CH3 and/or CH4 domain on each fusion polypeptide.

In some embodiments, at least two covalent bonds are located C-terminally to a CH2 and/or CH3 domain on each fusion polypeptide.

In some embodiments, at least two covalent bonds are located C-terminally to a CH2 and CH3 domain on each polypeptide.

Typically, the two or more covalent bonds which are located C-terminally to the portion of each fusion polypeptide which forms the Fc region.

In an embodiment of the provided insulin-Fc conjugate, the covalent bonds are disulfide bonds. Such disulfide bonds may be formed between thiol groups of two cysteine residues. Thus, the insulin Fc fusion polypeptide as referred to herein, typically, comprises at least one cysteine residue, such as two cysteine residues. In an embodiment, the cysteine residue or cysteine residues is (are) present within the C-terminal region of the fusion polypeptide, e.g. within the 20 C-terminal amino acids of the fusion polypeptide.

Typically, the two fusion polypeptides are not covalently linked by one or more disulfide bonds between the N-terminus of each IgG Fc region polypeptide.

Typically, each the first insulin-Fc fusion polypeptide and the second insulin Fc fusion polypeptide comprises at the C-terminus an amino acid sequence which comprises at least two cysteine residues allowing the formation of two disulfide bonds between the first and second fusion polypeptide (herein also referred to as “C-terminal peptide”). In this case, the at least two C-terminal covalent bonds are at least two disulfide bonds within this sequence.

The C-terminal peptide, e.g. may have a length of 7 to 20 amino acids, such as of 10 to 20 amino acids.

The amino acid sequence at the C-terminus of the fusion polypeptide provided herein (i.e. of the C-terminal peptide) can be the amino acid sequence of a naturally occurring antibody hinge region, or a sequence which is highly similar to said sequence, for example the sequence of the hinge region of an IgG1 or an IgG4 antibody (or a sequence which is highly similar to said sequence).

The amino acid sequence of the IgG1 heavy chain constant region is shown in SEQ ID NO: 35. The hinge region of this antibody can be found at position 99 to 110 of this sequence and has the following amino acid sequence: EPKSCDKTHTCP (SEQ ID NO: 36).

The amino acid sequence of the IgG4 heavy chain constant region is shown in SEQ ID NO: 38. The hinge region can be found at position 99 to 110 of this sequence and has the following amino acid sequence: ESKYGPPCPSCP (SEQ ID NO: 39). Further, the hinge region can be found at position 99 to 111 of this sequence and has the following amino acid sequence: ESKYGPPCPSCPA (SEQ ID NO: 40).

Thus, the C-terminal peptide present at the C-terminus may comprise or consist of an amino acid sequence as shown in SEQ ID NO: 36, 39 or 40.

In some embodiments, however, the C-terminal peptide present at the C-terminus has at least 70% identity (i.e. is highly similar) to the sequence of an antibody hinge region, i.e. naturally occurring antibody hinge region. In some embodiments, the sequence has at least 75% identity to the sequence of an antibody hinge region. In some embodiments, the sequence has at least 80% identity to the sequence of an antibody hinge region. In some embodiments, the sequence has at least 90% identity to the sequence of an antibody hinge region. In some embodiments, the sequence comprises at least 7 contiguous amino acids of the sequence of an antibody hinge region. In some embodiments, the sequence comprises at least 10 contiguous amino acids of the sequence of an antibody hinge region. In some embodiments, the sequence comprises at least 15 contiguous amino acids of the sequence of an antibody hinge region. Typically, the antibody hinge region comprises two or more cysteine residues (to form the disulfide bonds between the two fusion polypeptides).

As the sequence of the antibody hinge region can be derived from a human endogenous antibody hinge region, the usage of exogeneous sequences can be avoided which reduces the risk of anti-drug antibody (ADA) formation.

In the studies described herein the following peptide was used as C-terminal peptide ESKYGPPCPPCPA (SEQ ID NO: 41). The sequence of this peptide is highly similar to a naturally occurring antibody hinge region. Thus, the C-terminal peptide present at the C-terminus may comprise or consist of an amino acid sequence as shown in SEQ ID NO: 41.

In one embodiment, the Fc region and the C-terminal region are linked by a third linker peptide. Said third linker typically has a length of 1 to 10 amino acids, such as a length of 6 amino acids. In an embodiment, the third linker peptide comprises or consists of sequence as shown in GGGGSA (SEQ ID NO: 42).

Accordingly, the insulin-Fc fusion polypeptide provided herein comprises, in some embodiments, from N- to C-terminus:
a) an insulin B chain,
b) a first linker peptide,
c) an insulin A chain,
d) a second linker peptide,
e) an Fc region
d) a third linker peptide, and
e) a C-terminal peptide

Said C-terminal peptide shall allow the formation of at least two covalent bonds, i.e. inter-chain covalent bonds, to a further insulin-Fc fusion polypeptide (typically a fusion polypeptide having the same sequence). Thereby, a form stable dimer due to the C-terminal at least two inter-chain covalent bonds is formed. In some embodiments, the at least two covalent bonds are at least two disulfide bonds. In some embodiments, the at least two covalent bonds are two disulfide bonds.

Typically, said C-terminal peptide comprises the amino acid sequence of an antibody hinge region (or is a sequence which is highly similar thereto). In some embodiments, the said C-terminal peptide has an amino acid sequence as shown in SEQ ID NO: 36, 39, 40 or 41.

As set forth above, the Fc region, in some embodiments, lacks at the N-terminus the antibody hinge region. Accordingly, the fusion polypeptides—within the conjugate provided herein—are not linked by a disulfide bond located N-terminally to the portion of each polypeptide which forms the Fc region of the conjugate.

Accordingly, the two fusion polypeptides form an antibody Fc region,
wherein said two polypeptides are linked by at least two covalent bonds which are located C-terminally to the portion of each polypeptide which forms the Fc region,
wherein said two polypeptides are not linked by a disulfide bond located N-terminally to the portion of each polypeptide which forms the Fc region.

In an embodiment, the Fc region which lacks an antibody hinge region comprises or consists of
a) an amino acid sequence as shown in SEQ ID NO: 34 (GGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLPP SRDELTKNQVSLTCLVKGFYPSDIAVEWESNGQPENNYKTTPPVLDSDGSFFLYSKLTV DKSRWQQGNVFSCSVMHEALHNHYTQKSLSLSPG), or
b) a sequence being at least 90%, at least 91%, at least 92%, at least 93%, at least 94%, at least 95%, at least 96%, at least 97%, at least 98% or at least 99% identical to SEQ ID NO: 34.

SEQ ID NO: 34 is the Fc region of the human IgG1 antibody (without hinge and C-terminal Lys) and was used in many constructs described in Table A. It comprises the region from position 119 to 329 of human IgG1. The hinge as well the C-terminal Lys at position 330 are not included.

In another embodiment, the Fc region which lacks an antibody hinge region comprises or consists of
a) a sequence as shown in SEQ ID NO: 37, or
b) a sequence being at least 90%, at least 91%, at least 92%, at least 93%, at least 94%, at least 95%, at least 96%, at least 97%, at least 98% or at least 99% identical to SEQ ID NO: 37.

In some embodiments, an Fc region present within the fusion polypeptide provided herein comprises the following amino acid sequence:

(SEQ ID NO: 83)
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVD
KSRWQQGNVFSCSVMHEALHNHYTQKSLSLSPGGGGGSAESKYGPPCPPCPA.

The sequence which includes the third linker peptide and the C-terminal hinge region was used in the Examples section. The sequence of the third linker peptide is underlined. The C-terminal hinge region is highlighted in bold.

In some embodiments, an Fc region present within the fusion polypeptide provided herein comprises the following amino acid sequence:

(SEQ ID NO: 84)
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVD
KSRWQQGNVFSCSVMHEALHNHYTQKSLSLSPGAESKYGPPCPPCPA.

In some embodiments, an Fc region present within the fusion polypeptide provided herein comprises the following amino acid sequence:

(SEQ ID NO: 85)
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVD
KSRWQQGNVFSCSVMHEALHNHYTQKSLSLSPGGPPCPPCPA.

### Further Aspects (Host Cell, Polynucleotide, Medical Uses)

Further provided is a polynucleotide encoding for the single-chain insulin provided herein, or the insulin-Fc fusion polypeptide provided herein.

Further provided is a host cell comprising the insulin-Fc conjugate provided herein, the single-chain insulin provided herein, or the insulin-Fc fusion polypeptide provided, and/or the polynucleotide provided herein.

Further provided is a method of producing the insulin-Fc conjugate provided herein, the single-chain insulin provided herein, or the insulin-Fc fusion polypeptide provided herein, comprising incubating the host cell provided herein under conditions that allow for expressing the insulin-Fc conjugate, the single-chain insulin, or the insulin-Fc fusion polypeptide.

Further provided is a pharmaceutical composition comprising in a pharmaceutically effective amount the insulin-Fc conjugate provided herein, the single-chain insulin provided herein, or the insulin-Fc fusion polypeptide provided herein.

Further provided is a pharmaceutical composition comprising in a pharmaceutically effective amount insulin-Fc conjugate provided herein, the single-chain insulin provided herein, or the insulin-Fc fusion polypeptide provided herein.

Further provided is the insulin-Fc conjugate of provided herein, the insulin provided herein (such as the single chain insulin provided herein), or the insulin-Fc fusion polypeptide provided herein for use as a medicament.

Further provided is the insulin-Fc conjugate of provided herein, the insulin provided herein (such as the single chain insulin provided herein), or the insulin-Fc fusion polypeptide provided herein for use as a medicament for treatment of a disease selected from the group consisting of gestational diabetes, diabetes mellitus type 1, diabetes mellitus type 2, and hyperglycemia and/or for lowering blood glucose levels.

### Embodiments

In the following embodiments of the provided conjugate, fusion polypeptide and insulin are disclosed. The definitions and explanations given herein above apply mutatis mutandis the following.
1. An insulin-Fc conjugate of two insulin-Fc fusion polypeptides, wherein each of said two insulin-Fc fusion polypeptides comprises from N- to C-terminus: an insulin and an Fc region polypeptide.
2. The insulin-Fc conjugate of embodiment 1, wherein said two insulin-Fc fusion polypeptides are linked by at least two covalent bonds at the C-terminus of the fusion polypeptides, for example wherein the at least two covalent bonds are disulfide bonds.
3. The insulin-Fc conjugate of embodiment 1 or 2, wherein said conjugate is a homodimer of said two insulin-Fc fusion polypeptides and/or wherein the two insulin-Fc fusion polypeptides form an antibody Fc region.
4. The insulin-Fc conjugate of any one of the preceding embodiments, wherein the insulin is a single chain insulin.
5. The insulin-Fc conjugate of any one of the preceding embodiments, wherein each of the two insulin-Fc fusion polypeptides comprises from N- to C-terminus:
   a) an insulin B chain,
   b) a first linker peptide,
   c) an insulin A chain,
   d) a second linker peptide,
   e) an Fc region polypeptide
6. The insulin-Fc conjugate of embodiment 5, wherein each of the two insulin-Fc fusion polypeptides further comprises
   f) a third linker peptide,
   g) a C-terminal peptide allowing the formation of said at least two covalent bonds between said two insulin-Fc fusion polypeptides
7. The insulin-Fc conjugate of embodiment 6, wherein the C-terminal peptide under g) comprises at least two cysteine residues (such as two cysteine residues) allowing the formation of at least two disulfide bonds between the two insulin-Fc fusion polypeptides, for example wherein the C-terminal peptide comprises the amino acid sequence of an antibody hinge region comprising at least two cysteine residues allowing the formation of at least two disulfide bonds between the two insulin-Fc fusion polypeptides (or a highly similar sequence).
8. The insulin-Fc conjugate of embodiment 7, wherein the amino acid sequence of the C-terminal peptide comprises or consists of an amino acid sequence as shown in SEQ ID NO: 36, 39, 40 or 41.
9. The insulin-Fc conjugate of any one of the preceding embodiments, wherein said two insulin-Fc fusion polypeptides are not covalently linked by one or more disulfide bonds between the N-terminus of each Fc region polypeptide.
10. The insulin-Fc conjugate of any one of the preceding embodiments, wherein the Fc region polypeptide lacks an antibody hinge region at the N-terminus.
11. The insulin-Fc conjugate of any one of the preceding embodiments, wherein the Fc region polypeptide comprises the constant domains of an IgG, IgM, IgA, IgD or IgE antibody heavy chain.
12. The insulin-Fc conjugate of embodiment 12, wherein the Fc region polypeptide comprises the constant domains CH2 and CH3 of the antibody heavy chain an IgG1 antibody or an IgG4 antibody
13. The insulin-Fc conjugate of any one of the preceding embodiments, wherein the first linker peptide has a length of 1 to 50, such as 5 to 15 amino acids, such as 5 to 10 amino acids, such as 7 amino acids.
14. The insulin-Fc conjugate of embodiment 13, wherein the first linker peptide comprises or consists of the amino acid sequence GSYPGGV (SEQ ID NO: 26) or EEYPGDV (SEQ ID NO: 27).
15. The insulin-Fc conjugate of any one of the preceding embodiments, wherein said second linker peptide has a length of 1 to 100, such as of 5 to 40 amino acids.
16. The insulin-Fc conjugate of embodiment 15, wherein said second linker peptide is the human insulin C-peptide, or a fragment thereof.
17. The insulin-Fc conjugate of embodiment 16, wherein the said second linker comprises or consists of an amino acid sequence shown in SEQ ID NO: 28 or 29.
18. The insulin-Fc conjugate of any one of the preceding embodiments, wherein said third linker has a length of 1 to 10 amino acids, such as a length of 6 amino acids
19. The insulin-Fc conjugate of embodiment 18, wherein said third linker peptide comprises or consists of an amino acid sequence as shown in GGGGSA (SEQ ID NO: 42).
20. The insulin-Fc conjugate of any one of the preceding embodiments, wherein said insulin is an insulin having reduced binding affinity to the human insulin receptor as compared to the binding affinity of human insulin to human insulin receptor.
21. The insulin-Fc conjugate of any one of the preceding embodiments, wherein said insulin is an insulin shown in Table B, such as a single chain insulin shown in Table B.
22. The insulin-Fc conjugate of embodiment 21, wherein
   i) the insulin B chain comprises or consists of the amino acid sequence SFVNQHLCGSHLVEALELVCGERGFHYTPKT (SEQ ID NO: 69) and the insulin A chain comprises or consists of the amino acid sequence GIVEQCCTSICSLEQLENYCG (SEQ ID NO: 74),
   ii) the insulin B chain comprises or consists of the amino acid sequence SFVNQHLCGSHLVEALHLVCGERGFAYTPKT (SEQ ID NO: 70) and the insulin A chain comprises or consists of the amino acid sequence GIVEQCCTSICSLEQLENYCG (SEQ ID NO: 74),
   iii) the insulin B chain comprises or consists of the amino acid sequence FVNQHLCGSHLVEALHLVCGERGFHYTPKT (SEQ ID NO: 71) and the insulin A chain comprises or consists of the amino acid sequence GIVEQCCTSICSLSQLEDYCG (SEQ ID NO: 75),
   iv) the insulin B chain comprises or consists of the amino acid sequence SFVNQHLCGSHLVEALELVCGERGFHYTPKT (SEQ ID NO: 73) and the insulin A chain comprises or consists of the amino acid sequence GIVEQCCTSICSLSQLEDYCG (SEQ ID NO: 75),
   v) the insulin B chain comprises or consists of the amino acid sequence SFVNQHLCGSHLVEALYLVCGERGFFYTPKT (SEQ ID NO: 65) and the insulin A chain comprises or consists of the amino acid sequence GIVEQCCTSICSLEQLENYCG (SEQ ID NO: 74),
   vi) the insulin B chain comprises or consists of the amino acid sequence SFVNQHLCGSHLVEALHLVCGERGFAYTDKT (SEQ ID NO: 66) and the insulin A chain comprises or consists of the amino acid sequence GIVEQCCTSICSLEQLENYCG (SEQ ID NO: 74), or
   vii) the insulin B chain comprises or consists of the amino acid sequence SFVNQHLCGSHLVEALHLVCGERGFAYTDKT (SEQ ID NO: 67) and wherein the insulin A chain comprises or consists of the amino acid sequence GIVEQCCTSICSLEQLENYCG (SEQ ID NO: 74).
23. The insulin-Fc conjugate of embodiment 21 or 22, wherein said insulin is a single chain insulin comprising an amino acid sequence shown in SEQ ID NO: 50, SEQ ID NO: 52, SEQ ID NO: 53, SEQ ID NO: 58, SEQ ID NO: 45, SEQ ID NO: 47, SEQ ID NO: 81, or SEQ ID NO: 82.
24. The insulin-Fc conjugate of any one of the preceding embodiments, wherein each of said two insulin-Fc fusion polypeptides comprises an amino acid sequence shown in SEQ ID NO: 10, SEQ ID NO: 12, SEQ ID NO: 14, SEQ ID NO: 19, SEQ ID NO: 4, SEQ ID NO: 5, SEQ ID NO: 6, or SEQ ID NO: 7.
25. An insulin according to the definition of the insulin in embodiment 22 or 23, such as the single-chain insulin according to the definition of the insulin in embodiment 23.
26. An insulin-Fc fusion polypeptide according to the definition of the insulin-Fc fusion polypeptide in any one of embodiments 1 to 24.
27. A pharmaceutical composition comprising in a pharmaceutically effective amount insulin-Fc conjugate of any one of embodiments 1 to 24, the insulin of embodiment
25, or the insulin-Fc fusion polypeptide of embodiment 26. 28. The insulin-Fc conjugate of any one of embodiments 1 to 24, the insulin of embodiment 25, or the insulin-Fc fusion polypeptide of embodiment 26 for use as a medicament.
29. The insulin-Fc conjugate of any one of embodiments 1 to 24, the insulin of embodiment 25, or the insulin-Fc fusion polypeptide of embodiment 26 for use as a medicament for treatment of a disease selected from the group consisting of gestational diabetes, diabetes mellitus type 1, diabetes mellitus type 2, and hyperglycemia and/or for lowering blood glucose levels.
30. A polynucleotide encoding for the insulin of embodiment 25, or the insulin-Fc fusion polypeptide of embodiment 26.
31. A host cell comprising the insulin-Fc conjugate of any one of embodiments 1 to 24, the insulin of embodiment 25, or the insulin-Fc fusion polypeptide of embodiment 26, and/or the polynucleotide of embodiment 20.
32. A method of producing the insulin-Fc conjugate of any one of embodiments 1 to 24, the insulin of embodiment 25, or the insulin-Fc fusion polypeptide of embodiment 26, comprising incubating the host cell of embodiment 21 under conditions that allow for expressing the insulin-Fc conjugate of any one of embodiments 1 to 24, the insulin of embodiment 25, or the insulin-Fc fusion polypeptide of embodiment 26.

### EXAMPLES

### Methods

### Protein Expression and Purification

The scl-Fc proteins INS001-INS027 (SEQ ID NOs: 2 to 25, 77 to 79, see also Table A) were fused to a leader sequence (SEQ ID NO: 1) that directs protein expression into the culture supernatant. The scl-Fc proteins were produced by transient transfection either in HEK293 or CHO cells. The proteins were purified from the culture supernatant using protein A (mab select sure, GE Healthcare) affinity chromatography. After protein A purification the scl-Fc proteins were further purified using a gelfiltration column (Superdex 200, GE healthcare), equilibrated in phosphate buffered saline (PBS, Gibco). Fractions containing the desired protein were collected, pooled concentrated and stored at −80° C. until further usage.

### In Vitro Assay Insulin Receptor Phosphorylation

The produced scl-Fc proteins were subjected to insulin receptor phosphorylation assays. Receptor phosphorylation (herein also referred to as “autophosphorylation”) was measured following essentially the protocol described by Sommerfeld et al. (Sommerfeld et al., 2010). CHO cells expressing the corresponding insulin receptors using in cell Wester technology. CHO-IR cells were cultivated in Ham's F12-Nutrient Mix/GlutaMax medium (Gibco/Thermo Fisher scientific) containing 10% (v/v) fetal calf serum (PAN-Biotech), 1× nonessential amino acids (Gibco/Thermo Fisher scientific) and adequate antibiotics for selection. To determine receptor phosphorylation after insulin stimulation, cells were seeded into 96-well plates and grown for 44 h. Cells were serum starved with serum-free Ham's Nutrient Mixture F12 medium for 2 h and subsequently treated with increasing concentrations of insulin or scl-Fc-variant for 20 min at 37° C. after incubation, medium was discarded and cells were fixed in 3.75% freshly prepared paraformaldehyde for 20 min. Cells were permeabilized with 0.1% Triton X-100 in PBS (Gibco) for 20 min followed by blocking with Odyssey blocking buffer (LI-COR Biosciences) for 1 hr at ambient temperature. Anti-phosphotyrosine antibody 4G10 (Millipore) was used as primary antibody. After 2 h incubation at ambient temperature cells were washed 3 times with PS+0.1% Twen 20. The secondary anti-mouse IgG G-800-CW and DNA stain DRAQ5 (Cell Signaling), which was required for cell number normalization, were added and incubated for 1 h. Cells were washed 3 times with PBS+0.1% Tween 20 and fluorescence signals were at 700 and 800 nm were determined with a LI-COR Infrared imaging system (Odyssey, LI-COR Biosciences). Data were obtained at relative fluorescence units (RFU).

The single chain insulins present in the constructs shown in Table A were tested. The sequences of the single-chain insulins are also shown in Table B As control human insulin (B chain: SEQ ID NO: 32, A chain: SEQ ID NO: 33) was used.

TABLE A
Tested single chain insulin Fc (scl Fc) fusion polypeptides. The single chain insulin analog is
shown in bold italics, the hinge at the N-terminus in bold. All linker sequences (from N- to C-
terminus: first linker, second linker and third linker) are underlined. The column “comments
on insulin” contains information on the tested insulins. The modifications as compared to human
insulin are indicated. For example, “GluA14” means that the amino acid at position 14 of the A
chain of human insulin has been replaced with Glu. “SerB0” means that a serine residue was
added at the N-terminus of the B chain. “GlyA21” means that a glycine residue was added at the
C-terminus of the A chain. The sequence of human insulin is disclosed elsewhere herein.

| SCI-Fc | | | |
|---|---|---|---|
| fusion | | | |
| protein | | SEQ ID | |
| No. | Sequence | NO | Comments on insulin |
| INS001 | FVNQHLCGSHLVEALYLVCGERGFFYTPKTEEYPGDVGIVEQCCTSICSLEQLENY | 2 | GluA14_GlyA21 |
| | CGGEAEDLQVGQVELGGGGPSVFLFPPKPKDTLMISRTPEVTCVVVDVSHEDPEVK | | |
| | FNWYVDGVEVHNAKTKPREEQYNSTYRVVSVLTVLHQDWLNGKEYKCKVSNKALPA | | |
| | PIEKTISKAKGQPREPQVYTLPPSRDELTKNQVSLTCLVKGFYPSDIAVEWESNGQ | | |
| | PENNYKTTPPVLDSDGSFFLYSKLTVDKSRWQQGNVFSCSVMHEALHNHYTQKSLS | | |
| | LSPGGGGGSAESKYGPPCPPCPA | | |
| INS002 | FVNQHLCGSHLVEALYLVCGERGFFYTDKTEEYPGDVGIVEQCCTSICSLEQLENY | 3 | AspB28_GluA14_ |
| | CGGEAEDLQVGQVELGGGPGAGSLQPLALEGSLQGGPSVFLFPPKPKDTLMISRTP | | GlyA21 |
| | EVTCVVVDVSHEDPEVKFNWYVDGVEVHNAKTKPREEQYNSTYRVVSVLTVLHQDW | | |
| | LNGKEYKCKVSNKALPAPIEKTISKAKGQPREPQVYTLPPSRDELTKNQVSLTCLV | | |
| | KGFYPSDIAVEWESNGQPENNYKTTPPVLDSDGSFFLYSKLTVDKSRWQQGNVFSC | | |
| | SVMHEALHNHYTQKSLSLSPGGGGGSAESKYGPPCPPCPA | | |
| INS003 | SEVNQHLCGSHLVEALYLVCGERGFFYTPKTEEYPGDVGIVEQCCTSICSLEQLEN | 4 | SerB0_InsA- |
| | YCGGEAEDLQVGQVELGGGPGAGSLQPEGSLQGGPSVFLFPPKPKDTLMISRTPEV | | GluA14_GlyA21 |
| | TCVVVDVSHEDPEVKFNWYVDGVEVHNAKTKPREEQYNSTYRVVSVLTVLHQDWLN | | |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS004 | SFVNQHLCGSHLVEALYLVCGERGFFYTPKTGSYPGGVGIVEQCCTSICSLEQLEN | 5 | SerB0_GluA14_ |
| | YCGGEAEDLQVGQVELGGGPGAGSLQPEGSLQGGPSVFLFPPKPKDTLMISRTPEV | | GlyA21 |
| | TCVVVDVSHEDPEVKFNWYVDGVEVHNAKTKPREEQYNSTYRVVSVLTVLHQDWLN | | |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS005 | SFVNQHLCGSHLVEALHLVCGERGFAYTDKTEEYPGDVGIVEQCCTSICSLEQLEN | 6 | SerB0_HisB16_ |
| | YCGGEAEDLQVGQVELGGGPGAGSLQPEGSLQGGPSVFLFPPKPKDTLMISRTPEV | | AlaB25_AspB28_ |
| | TCVVVDVSHEDPEVKFNWYVDGVEVHNAKTKPREEQYNSTYRVVSVLTVLHQDWLN | | GluA14_GlyA21 |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS006 | SFVNQHLCGSHLVEALELVCGERGFHYTDKTEEYPGDVGIVEQCCTSICSLEQLEN | 7 | SerB0_GluB16_ |
| | YCGGEAEDLQVGQVELGGGPGAGSLQPEGSLQGGPSVFLFPPKPKDTLMISRTPEV | | HisB25_AspB28_ |
| | TCVVVDVSHEDPEVKFNWYVDGVEVHNAKTKPREEQYNSTYRVVSVLTVLHQDWLN | | GluA14_GlyA21 |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS007 | SFVNQHLCGSHLVEALHLVCGERGFAYTDKTGSYPGGVGIVEQCCTSICSLEQLEN | 8 | SerB0_HisB16_ |
| | YCGGEAEDLQVGQVELGGGPGAGSLQPEGSLQGGPSVFLFPPKPKDTLMISRTPEV | | AlaB25_AspB28_ |
| | TCVVVDVSHEDPEVKFNWYVDGVEVHNAKTKPREEQYNSTYRVVSVLTVLHQDWLN | | GluA14_GlyA21 |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS008 | SFVNQHLCGSHLVEALELVCGERGFHYTDKTGSYPGGVGIVEQCCTSICSLEQLEN | 9 | SerB0_GluB16- |
| | YCGGEAEDLQVGQVELGGGPGAGSLQPEGSLQGGPSVFLFPPKPKDTLMISRTPEV | | HisB25_AspB28_ |
| | TCVVVDVSHEDPEVKFNWYVDGVEVHNAKTKPREEQYNSTYRVVSVLTVLHQDWLN | | GluA14_GlyA21 |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS009 | SFVNQHLCGSHLVEALELVCGERGFHYTPKTGSYPGGVGIVEQCCTSICSLEQLEN | 10 | SerB0_GluB16_ |
| | YCGGEAEDLQVGQVELGGGPGAGSLQPEGSLQGGPSVFLFPPKPKDTLMISRTPEV | | HisB25-GluA14_GlyA21 |
| | TCVVVDVSHEDPEVKFNWYVDGVEVHNAKTKPREEQYNSTYRVVSVLTVLHQDWLN | | |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS010 | SFVNQHLCGSHLVEALHLVCGERGFAYTPKTEEYPGDVGIVEQCCTSICSLEQLEN | 11 | SerB0_HisB16_ |
| | YCGGEAEDLQVGQVELGGGPGAGSLQPEGSLQGGPSVFLFPPKPKDTLMISRTPEV | | AlaB25_GluA14_GlyA21 |
| | TCVVVDVSHEDPEVKFNWYVDGVEVHNAKTKPREEQYNSTYRVVSVLTVLHQDWLN | | |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS011 | SFVNQHLCGSHLVEALHLVCGERGFAYTPKTGSYPGGVGIVEQCCTSICSLEQLEN | 12 | SerB0_HisB16_ |
| | YCGGEAEDLQVGQVELGGGPGAGSLQPEGSLQGGPSVFLFPPKPKDTLMISRTPEV | | AlaB25_GluA14_GlyA21 |
| | TCVVVDVSHEDPEVKFNWYVDGVEVHNAKTKPREEQYNSTYRVVSVLTVLHQDWLN | | |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS012 | FVNQHLCGSHLVEALHLVCGERGFHYTPKTEEYPGDVGIVEQCCTSICSLSQLEDY | 13 | HisB16_HisB25_ |
| | CGGEAEDLQVGQVELGGGPGAGSLQPEGSLQGGPSVFLFPPKPKDTLMISRTPEVT | | SerA14_AspA18_GlyA21 |
| | CVVVDVSHEDPEVKFNWYVDGVEVHNAKTKPREEQYNSTYRVVSVLTVLHQDWLNG | | |
| | KEYKCKVSNKALPAPIEKTISKAKGQPREPQVYTLPPSRDELTKNQVSLTCLVKGF | | |
| | YPSDIAVEWESNGQPENNYKTTPPVLDSDGSFFLYSKLTVDKSRWQQGNVFSCSVM | | |
| | HEALHNHYTQKSLSLSPGGGGGSAESKYGPPCPPCPA | | |
| INS013 | FVNQHLCGSHLVEALHLVCGERGFHYTPKTGSYPGGVGIVEQCCTSICSLSQLEDY | 14 | HisB16_HisB25_ |
| | CGGEAEDLQVGQVELGGGPGAGSLQPEGSLQGGPSVFLFPPKPKDTLMISRTPEVT | | SerA14_AspA18_GlyA21 |
| | CVVVDVSHEDPEVKFNWYVDGVEVHNAKTKPREEQYNSTYRVVSVLTVLHQDWLNG | | |
| | KEYKCKVSNKALPAPIEKTISKAKGQPREPQVYTLPPSRDELTKNQVSLTCLVKGF | | |
| | YPSDIAVEWESNGQPENNYKTTPPVLDSDGSFFLYSKLTVDKSRWQQGNVFSCSVM | | |
| | HEALHNHYTQKSLSLSPGGGGGSAESKYGPPCPPCPA | | |
| INS014 | SFVNQHLCGSHLVEALHLVCGERGFHYTPKTGSYPGGVGIVEQCCTSICSLSQLED | 15 | SerB0_HisB16_ |
| | YCGGEAEDLQVGQVELGGGPGAGSLQPEGSLQGGPSVFLFPPKPKDTLMISRTPEV | | HisB25_SerA14_ |
| | TCVVVDVSHEDPEVKFNWYVDGVEVHNAKTKPREEQYNSTYRVVSVLTVLHQDWLN | | AspA18_GlyA21 |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS015 | SFVNQHLCGSHLVEALHLVCGERGFHYTPKTEEYPGDVGIVEQCCTSICSLSQLEN | 16 | SerB0_HisB16_ |
| | YCGGEAEDLQVGQVELGGGPGAGSLQPEGSLQGGPSVFLFPPKPKDTLMISRTPEV | | HisB25_SerA14_GlyA21 |
| | TCVVVDVSHEDPEVKFNWYVDGVEVHNAKTKPREEQYNSTYRVVSVLTVLHQDWLN | | |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS016 | FVNQHLCGSHLVEALHLVCGERGFHYTPKTGSYPGGVGIVEQCCTSICSLSQLENY | 17 | HisB16_HisB25_ |
| | CGGEAEDLQVGQVELGGGPGAGSLQPEGSLQGGPSVFLFPPKPKDTLMISRTPEVT | | SerA14_GlyA21_ |
| | CVVVDVSHEDPEVKFNWYVDGVEVHNAKTKPREEQYNSTYRVVSVLTVLHQDWING | | |
| | KEYKCKVSNKALPAPIEKTISKAKGQPREPQVYTLPPSRDELTKNQVSLTCLVKGF | | |
| | YPSDIAVEWESNGQPENNYKTTPPVLDSDGSFFLYSKLTVDKSRWQQGNVFSCSVM | | |
| | HEALHNHYTQKSLSLSPGGGGGSAESKYGPPCPPCPA | | |
| INS017 | SFVNQHLCGSHLVEALHLVCGERGFHYTPKTGSYPGGVGIVEQCCTSICSLSQLEN | 18 | SerB0_HisB16_ |
| | YCGGEAEDLQVGQVELGGGPGAGSLQPEGSLQGGPSVFLFPPKPKDTLMISRTPEV | | HisB25_SerA14_GlyA21 |
| | TCVVVDVSHEDPEVKFNWYVDGVEVHNAKTKPREEQYNSTYRVVSVLTVLHQDWLN | | |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS018 | FVNQHLCGSHLVEALELVCGERGFHYTPKTGSYPGGVGIVEQCCTSICSLSQLEDY | 19 | GluB16_HisB25_ |
| | CGGEAEDLQVGQVELGGGPGAGSLQPEGSLQGGPSVFLFPPKPKDTLMISRTPEVT | | SerA14_AspA18_GlyA21 |
| | CVVVDVSHEDPEVKFNWYVDGVEVHNAKTKPREEQYNSTYRVVSVLTVLHQDWING | | |
| | KEYKCKVSNKALPAPIEKTISKAKGQPREPQVYTLPPSRDELTKNQVSLTCLVKGF | | |
| | YPSDIAVEWESNGQPENNYKTTPPVLDSDGSFFLYSKLTVDKSRWQQGNVFSCSVM | | |
| | HEALHNHYTQKSLSLSPGGGGGSAESKYGPPCPPCPA | | |
| INS019 | SFVNQHLCGSHLVEALELVCGERGFHYTPKTGSYPGGVGIVEQCCTSICSLSQLED | 20 | SerB0_GluB16_ |
| | YCGGEAEDLQVGQVELGGGPGAGSLQPEGSLQGGPSVFLFPPKPKDTLMISRTPEV | | HisB25_SerA14_ |
| | TCVVVDVSHEDPEVKFNWYVDGVEVHNAKTKPREEQYNSTYRVVSVLTVLHQDWLN | | AspA18_GlyA21 |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS020 | FVNQHLCGSHLVEALELVCGERGFHYTPKTEEYPGDVGIVEQCCTSICSLSQLENY | 21 | GluB16_HisB25_ |
| | CGGEAEDLQVGQVELGGGPGAGSLQPEGSLQGGPSVFLFPPKPKDTLMISRTPEVT | | SerA14_GlyA21 |
| | CVVVDVSHEDPEVKFNWYVDGVEVHNAKTKPREEQYNSTYRVVSVLTVLHQDWING | | |
| | KEYKCKVSNKALPAPIEKTISKAKGQPREPQVYTLPPSRDELTKNQVSLTCLVKGF | | |
| | YPSDIAVEWESNGQPENNYKTTPPVLDSDGSFFLYSKLTVDKSRWQQGNVFSCSVM | | |
| | HEALHNHYTQKSLSLSPGGGGGSAESKYGPPCPPCPA | | |
| INS021 | SEVNQHLCGSHLVEALELVCGERGFHYTPKTEEYPGDVGIVEQCCTSICSLSQLEN | 22 | SerB0_GluB16_ |
| | YCGGEAEDLQVGQVELGGGPGAGSLQLALEGSLQGGPSVFLFPPKPKDTLMISRTP | | HisB25_SerA14_GlyA21 |
| | EVTCVVVDVSHEDPEVKFNWYVDGVEVHNAKTKPREEQYNSTYRVVSVLTVLHQDW | | |
| | LNGKEYKCKVSNKALPAPIEKTISKAKGQPREPQVYTLPPSRDELTKNQVSLTCLV | | |
| | KGFYPSDIAVEWESNGQPENNYKTTPPVLDSDGSFFLYSKLTVDKSRWQQGNVFSC | | |
| | SVMHEALHNHYTQKSLSLSPGGGGGSAESKYGPPCPPCPA | | |
| INS022 | FVNQHLCGSHLVEALELVCGERGFHYTPKTGSYPGGVGIVEQCCTSICSLSQLENY | 23 | GluB16_HisB25_ |
| | CGGEAEDLQVGQVELGGGPGAGSLQPEGSLQGGPSVFLFPPKPKDTLMISRTPEVT | | SerA14_GlyA21 |
| | CVVVDVSHEDPEVKFNWYVDGVEVHNAKTKPREEQYNSTYRVVSVLTVLHQDWLNG | | |
| | KEYKCKVSNKALPAPIEKTISKAKGQPREPQVYTLPPSRDELTKNQVSLTCLVKGF | | |
| | YPSDIAVEWESNGQPENNYKTTPPVLDSDGSFFLYSKLTVDKSRWQQGNVFSCSVM | | |
| | HEALHNHYTQKSLSLSPGGGGGSAESKYGPPCPPCPA | | |
| INS023 | SFVNQHLCGSHLVEALELVCGERGFHYTPKTGSYPGGVGIVEQCCTSICSLSQLEN | 24 | SerB0_GluB16_ |
| | YCGGEAEDLQVGQVELGGGPGAGSLQPEGSLQGGPSVFLFPPKPKDTLMISRTPEV | | HisB25_-SerA14_GlyA21 |
| | TCVVVDVSHEDPEVKFNWYVDGVEVHNAKTKPREEQYNSTYRVVSVLTVLHQDWLN | | |
| | GKEYKCKVSNKALPAPIEKTISKAKGQPREPQVYTLPPSRDELTKNQVSLTCLVKG | | |
| | FYPSDIAVEWESNGQPENNYKTTPPVLDSDGSFFLYSKLTVDKSRWQQGNVFSCSV | | |
| | MHEALHNHYTQKSLSLSPGGGGGSAESKYGPPCPPCPA | | |
| INS024* | SFVNQHLCGSHLVEALELVCGERGFHYTPKTGSYPGGVGIVEQCCTSICSLEQLEN | 25 | SerB0_GluB16_ |
| | YCGGEAEDLQVGQVELGGGPGAGSLQPEGSLQDKTHTCPPCPAPEAAGGPSVFLFP | | HisB25_GluA14_GlyA21 |
| | PKPKDTLMISRTPEVTCVVVDVSHEDPEVKFNWYVDGVEVHNAKTKPREEQYNSTY | | |
| | RVVSVLTVLHQDWLNGKEYKCKVSNKALPAPIEKTISKAKGQPREPQVYTLPPSRD | | |
| | ELTKNQVSLTCLVKGFYPSDIAVEWESNGQPENNYKTTPPVLDSDGSFFLYSKLTV | | |
| | DKSRWQQGNVFSCSVMHEALHNHYTQKSLSLSPG | | |
| INS025** | FVNQHLCGSHLVEALELVCGERGFHYGGGGGGSGGGGGIVEQCCTSTCSLDQLENY | 77 | GluB16, HisB25, |
| | CGGGGGQGGGGQGGGGQGGGGGECPPCPAPPVAGPSVFLFPPKPKDTLMISRTPEV | | GlyB27, GlyB28, |
| | TCVVVDVSHEDPEVQFNWYVDGVEVHNAKTKPREEQFNSTFRVVSVLTVVHQDWLN | | GlyB29, GlyB30, |
| | GKEYKCKVSNKGLPAPIEKTISKTKGQPREPQVYTLPPSREEMTKNQVSLTCLVKG | | ThrA10, AspA14, |
| | FYPSDIAVEWESNGQPENNYKTTPPMLDSDGSFFLYSKLTVDKSRWQQGNVFSCSV | | Gly21 |
| | MHEALHNHYTQKSLSLSPG | | |
| INS026 | FVNQHLCGSHLVEALELVCGERGFHYGGGGGGSGGGGGIVEQCCTSTCSLDQLENY | 78 | GluB16, HisB25, |
| | CGGEAEDLQVGQVELGGGPGAGSLQPEGSLQGGPSVFLFPPKPKDTLMISRTPEVT | | GlyB27, GlyB28, |
| | CVVVDVSHEDPEVKFNWYVDGVEVHNAKTKPREEQYNSTYRVVSVLTVLHQDWLNG | | GlyB29, GlyB30, |
| | KEYKCKVSNKALPAPIEKTISKAKGQPREPQVYTLPPSRDELTKNQVSLTCLVKGF | | ThrA10, AspA14, |
| | YPSDIAVEWESNGQPENNYKTTPPVLDSDGSFFLYSKLTVDKSRWQQGNVFSCSVM | | Gly21 |
| | HEALHNHYTQKSLSLSPGGGGGSAESKYGPPCPPCPA | | |
| INS027 | FVNQHLCGSHLVEALELVCGERGFHYGGGGGGSGGGGGIVEQCCTSTCSLDQLENY | 79 | GluB16, HisB25, |
| | CGGGGGQGGGGQGGGGQGGGGGGGPSVFLFPPKPKDTLMISRTPEVTCVVVDVSHE | | GlyB27, GlyB28, |
| | DPEVKFNWYVDGVEVHNAKTKPREEQYNSTYRVVSVLTVLHQDWLNGKEYKCKVSN | | GlyB29, GlyB30, |
| | KALPAPIEKTISKAKGQPREPQVYTLPPSRDELTKNQVSLTCLVKGFYPSDIAVEW | | ThrA10, AspA14, |
| | ESNGQPENNYKTTPPVLDSDGSFFLYSKLTVDKSRWQQGNVFSCSVMHEALHNHYT | | GlyA21 |
| | QKSLSLSPGGGGGSAESKYGPPCPPCPA | | |

*does not comprise a hinge region at the C-terminus
** not a fusion polypeptide of the invention, disclosed in WO2016/178905

TABLE B
Sequence of i) the B chain and the A chain and ii) of the single chain insulin analogs of the
fusion proteins shown in Table A. The linker sequence is underlined in the single chain analog
is underlined.

| Present | | | |
|---|---|---|---|
| in fusion | | | |
| poly- | Sequence of the B chain | Sequence of the single | |
| peptide | and the A chain | chain insulin | Comments on insulin |
| INS001 | B chain: | FVNQHLCGSHLVEALYLVCGERGFFYTPKTE | GluA14_GlyA21 |
| | FVNQHLCGSHLVEALYLVCGERGFF | EYPGDVGIVEQCCTSICSLEQLENYCG (SEQ | |
| | YTPKT (SEQ ID NO: 32) | ID NO: 43) | |
| | A chain: | | |
| | GIVEQCCTSICSLEQLENYCG (SEQ | | |
| | ID NO: 74) | | |
| INS002 | B chain: | FVNQHLCGSHLVEALYLVCGERGFFYTDKTE | AspB28_GluA14_ |
| | FVNQHLCGSHLVEALYLVCGERGFF | EYPGDVGIVEQCCTSICSLEQLENYCG (SEQ | GlyA21 |
| | YTDKT (SEQ ID NO: 64) | ID NO: 44) | |
| | A chain: | | |
| | GIVEQCCTSICSLEQLENYCG (SEQ | | |
| | ID NO: 74) | | |
| INS003 | B chain: | SFVNQHLCGSHLVEALYLVCGERGFFYTPKT | SerB0_InsA- |
| | SFVNQHLCGSHLVEALYLVCGERGF | EEYPGDVGIVEQCCTSICSLEQLENYCG | GluA14_GlyA21 |
| | FYTPKT (SEQ ID NO: 65) | (SEQ ID NO: 45) | |
| | A chain: | | |
| | GIVEQCCTSICSLEQLENYCG (SEQ | | |
| | ID NO: 74) | | |
| INS004 | B chain: | SFVNQHLCGSHLVEALYLVCGERGFFYTPKT | SerB0_GluA14_ |
| | SFVNQHLCGSHLVEALYLVCGERGF | GSYPGGVGIVEQCCTSICSLEQLENYCG | GlyA21 |
| | FYTPKT (SEQ ID NO: 65) | (SEQ ID NO: 81) | |
| | A chain: | | |
| | GIVEQCCTSICSLEQLENYCG (SEQ | | |
| | ID NO: 74) | | |
| INS005 | B chain: | SFVNQHLCGSHLVEALHLVCGERGFAYTDKT | SerB0_HisB16_ |
| | SFVNQHLCGSHLVEALHLVCGERGF | EEYPGDVGIVEQCCTSICSLEQLENYCG | AlaB25_AspB28_ |
| | AYTDKT (SEQ ID NO: 66) | (SEQ ID NO: 82) | GluA14_GlyA21 |
| | A chain: | | |
| | GIVEQCCTSICSLEQLENYCG (SEQ | | |
| | ID NO: 74) | | |
| INS006 | B chain: | SFVNQHLCGSHLVEALELVCGERGFHYTDKT | SerB0_GluB16_ |
| | SFVNQHLCGSHLVEALHLVCGERGF | EEYPGDVGIVEQCCTSICSLEQLENYCG | HisB25_AspB28_ |
| | AYTDKT (SEQ ID NO: 67) | (SEQ ID NO: 47) | GluA14_GlyA21 |
| | A chain: | | |
| | GIVEQCCTSICSLEQLENYCG (SEQ | | |
| | ID NO: 74) | | |
| INS007 | B chain: | SFVNQHLCGSHLVEALHLVCGERGFAYTDKT | SerB0_HisB16_ |
| | SFVNQHLCGSHLVEALHLVCGERGF | GSYPGGVGIVEQCCTSICSLEQLENYCG | AlaB25_AspB28_ |
| | AYTDKT (SEQ ID NO: 66) | (SEQ ID NO: 48) | GluA14_GlyA21 |
| | A chain: | | |
| | GIVEQCCTSICSLEQLENYCG (SEQ | | |
| | ID NO: 74) | | |
| INS008 | B chain: | SFVNQHLCGSHLVEALELVCGERGFHYTDKT | SerB0_GluB16_ |
| | SFVNQHLCGSHLVEALHLVCGERGF | GSYPGGVGIVEQCCTSICSLEQLENYCG | HisB25_AspB28_ |
| | AYTDKT (SEQ ID NO: 67) | (SEQ ID NO: 49) | GluA14_GlyA21 |
| | A chain: | | |
| | GIVEQCCTSICSLEQLENYCG (SEQ | | |
| | ID NO: 74) | | |
| INS009 | B chain: | SFVNQHLCGSHLVEALELVCGERGFHYTPKT | SerB0_GluB16_ |
| | SFVNQHLCGSHLVEALELVCGERGF | GSYPGGVGIVEQCCTSICSLEQLENYCG | HisB25_-GluA14_ |
| | HYTPKT(SEQ ID NO: 73) | (SEQ ID NO: 50) | GlyA21 |
| | A chain: | | |
| | GIVEQCCTSICSLEQLENYCG (SEQ | | |
| | ID NO: 74) | | |
| INS010 | B chain: | SFVNQHLCGSHLVEALHLVCGERGFAYTPKT | SerB0_HisB16_AlaB25_ |
| | SFVNQHLCGSHLVEALHLVCGERGF | EEYPGDVGIVEQCCTSICSLEQLENYCG | GluA14_GlyA21 |
| | AYTPKT (SEQ ID NO: 70) | (SEQ ID NO: 51) | |
| | A chain: | | |
| | GIVEQCCTSICSLEQLENYCG (SEQ | | |
| | ID NO: 74) | | |
| INS011 | B chain: | SFVNQHLCGSHLVEALHLVCGERGFAYTPKT | SerB0_HisB16_ |
| | SFVNQHLCGSHLVEALHLVCGERGF | GSYPGGVGIVEQCCTSICSLEQLENYCG | AlaB25_GluA14_ |
| | AYTPKT (SEQ ID NO: 70) | (SEQ ID NO: 52) | GlyA21 |
| | A chain: | | |
| | GIVEQCCTSICSLEQLENYCG (SEQ | | |
| | ID NO: 74) | | |
| INS012 | B chain: | FVNQHLCGSHLVEALHLVCGERGFHYTPKTE | HisB16_HisB25_ |
| | FVNQHLCGSHLVEALHLVCGERGFH | EYPGDVGIVEQCCTSICSLSQLEDYCG (SEQ | SerA14_AspA18_ |
| | YTPKT (SEQ ID NO: 71) | ID NO: 80) | GlyA21 |
| | A chain: | | |
| | GIVEQCCTSICSLSQLEDYCG (SEQ | | |
| | ID NO: 75) | | |
| INS013 | B chain: | FVNQHLCGSHLVEALHLVCGERGFHYTPKTG | HisB16_HisB25_ |
| | FVNQHLCGSHLVEALHLVCGERGFH | SYPGGVGIVEQCCTSICSLSQLEDYCG (SEQ | SerA14_AspA18_ |
| | YTPKT (SEQ ID NO: 71) | ID NO: 53) | GlyA21 |
| | A chain: | | |
| | GIVEQCCTSICSLSQLEDYCG (SEQ | | |
| | ID NO: 75) | | |
| INS014 | B chain: | SFVNQHLCGSHLVEALHLVCGERGFHYTPKT | SerB0_HisB16_ |
| | SFVNQHLCGSHLVEALHLVCGERGF | GSYPGGVGIVEQCCTSICSLSQLEDYCG | HisB25_SerA14_ |
| | HYTPKT (SEQ ID NO: 86) | (SEQ ID NO: 54) | AspA18_GlyA21 |
| | A chain: | | |
| | GIVEQCCTSICSLSQLEDYCG (SEQ | | |
| | ID NO: 75) | | |
| INS015 | B chain: | SFVNQHLCGSHLVEALHLVCGERGFHYTPKT | SerB0_HisB16_ |
| | SFVNQHLCGSHLVEALHLVCGERGF | EEYPGDVGIVEQCCTSICSLSQLENYCG | HisB25_SerA14_ |
| | HYTPKT (SEQ ID NO: 86) | (SEQ ID NO: 55) | GlyA21 |
| | A chain | | |
| | GIVEQCCTSICSLSQLENYCG | | |
| | (SEQ ID NO: 76) | | |
| INS016 | B chain: | FVNQHLCGSHLVEALHLVCGERGFHYTPKTG | HisB16_HisB25_ |
| | FVNQHLCGSHLVEALHLVCGERGFH | SYPGGVGIVEQCCTSICSLSQLENYCG (SEQ | SerA14_GlyA21 |
| | YTPKT (SEQ ID NO: 71) | ID NO: 56) | |
| | A chain: | | |
| | GIVEQCCTSICSLSQLENYCG (SEQ | | |
| | ID NO: 76) | | |
| INS017 | B chain: | SFVNQHLCGSHLVEALHLVCGERGFHYTPKT | SerB0_HisB16_ |
| | SFVNQHLCGSHLVEALHLVCGERGF | GSYPGGVGIVEQCCTSICSLSQLENYCG | HisB25_SerA14_ |
| | HYTPKT (SEQ ID NO: 86) | (SEQ ID NO: 57) | GlyA21 |
| | A chain: | | |
| | GIVEQCCTSICSLSQLENYCG (SEQ | | |
| | ID NO: 76) | | |
| INS018 | B chain: | FVNQHLCGSHLVEALELVCGERGFHYTPKTG | GluB16_HisB25_ |
| | FVNQHLCGSHLVEALELVCGERGFH | SYPGGVGIVEQCCTSICSLSQLEDYCG (SEQ | SerA14_AspA18_ |
| | YTPKT (SEQ ID NO: 72) | ID NO: 58) | GlyA21 |
| | A chain: | | |
| | GIVEQCCTSICSLEQLENYCG (SEQ | | |
| | ID NO: 75) | | |
| INS019 | B chain: | SFVNQHLCGSHLVEALELVCGERGFHYTPKT | SerB0_GluB16_ |
| | SFVNQHLCGSHLVEALELVCGERGF | GSYPGGVGIVEQCCTSICSLSQLEDYCG | HisB25_SerA14_ |
| | HYTPKT (SEQ ID NO: 73) | (SEQ ID NO: 59) | AspA18_GlyA21 |
| | A chain: | | |
| | GIVEQCCTSICSLEQLENYCG (SEQ | | |
| | ID NO: 75) | | |
| INS020 | B chain: | FVNQHLCGSHLVEALELVCGERGFHYTPKTE | GluB16_HisB25_ |
| | FVNQHLCGSHLVEALELVCGERGFH | EYPGDVGIVEQCCTSICSLSQLENYCG (SEQ | SerA14_GlyA21 |
| | YTPKT (SEQ ID NO: 72) | ID NO: 60) | |
| | A chain: | | |
| | GIVEQCCTSICSLSQLENYCG (SEQ | | |
| | ID NO: 76) | | |
| INS021 | B chain: | SFVNQHLCGSHLVEALELVCGERGFHYTPKT | SerB0_GluB16_ |
| | SFVNQHLCGSHLVEALELVCGERGF | EEYPGDVGIVEQCCTSICSLSQLENYCG | HisB25_SerA14_ |
| | HYTPKT (SEQ ID NO: 73) | (SEQ ID NO: 61) | GlyA21 |
| | A chain: | | |
| | GIVEQCCTSICSLSQLENYCG (SEQ | | |
| | ID NO: 76) | | |
| INS022 | B chain: | FVNQHLCGSHLVEALELVCGERGFHYTPKTG | GluB16_HisB25_ |
| | FVNQHLCGSHLVEALELVCGERGFH | SYPGGVGIVEQCCTSICSLSQLENYCG (SEQ | SerA14_GlyA21 |
| | YTPKT (SEQ ID NO: 72) | ID NO: 62) | |
| | A chain: | | |
| | GIVEQCCTSICSLSQLENYCG (SEQ | | |
| | ID NO: 76) | | |
| INS023 | B chain: | SFVNQHLCGSHLVEALELVCGERGFHYTPKT | SerBO_GluB16_ |
| | SFVNQHLCGSHLVEALELVCGERGF | GSYPGGVGIVEQCCTSICSLSQLENYCG | HisB25_- |
| | HYTPKT (SEQ ID NO: 73) | (SEQ ID NO: 63) | SerA14_GlyA21 |
| | A chain: | | |
| | GIVEQCCTSICSLSQLENYCG (SEQ | | |
| | ID NO: 76) | | |
| INS024 | B chain: | SFVNQHLCGSHLVEALELVCGERGFHYTPKT | SerBO_GluB16_ |
| | SFVNQHLCGSHLVEALELVCGERGF | GSYPGGVGIVEQCCTSICSLEQLENYCG | HisB25_ |
| | HYTPKT (SEQ ID NO: 73) | (SEQ ID NO: 50) | GluA14_GlyA21 |
| | A chain: | | |
| | GIVEQCCTSICSLEQLENYCG (SEQ | | |
| | ID NO: 76) | | |

### In Vivo Rat PK/PD

Pharmacodynamic data were collected essentially as described by Faust et al. (Faust et al., 2020). Male Sprague-Dawly rats (Charles River) weighing 320-400 g at study start were randomized to treatment group of 8 with free access to tap water and food ad libitum. Proteins were dissolved in PBS (Gibco) and administered subcutaneously. PBS only was used as placebo control. Blood glucose was determined from whole blood taken from the tail tip at indicated time points. Blood was haemolyzed with haemolysate (haemolysis reagent H, glucose hexokinase fluid 5+1, Hengler analytic) and quantification was done with a Gluco-quant Glucose/Hexokinase kit (Roche Dagnostics) using a Beckman Coulter AU640 chemistry analyzer.

### In Vivo Minipig PK/PD

Healthy female Göttingen minipigs (Ellegaard Göttingen Minipigs) (aged 15-18 month, body weight ~20-25 kg) were used to evaluate pharmacodynamic and pharmacokinetics effects of long acting scl-Fc proteins. Animals were kept under standard animal house conditions and were fed once daily with access to tap water ad libitum.

Overnight fasted animals were given a single subcutaneous injection containing either placebo (PBS) or the corresponding scl-Fc variant. Blood was collected via a pre-implanted central venous catheter. The first blood sample was taken before the first administration, serving as baseline. At given timepoints blood was collected (1-4 times per day during the time of the study, K-EDTA containing sample tubes), stored immediately on ice and after centrifugation blood plasma was stored at −80° C. until further usage. Blood glucose was quantified from hemolyzed samples (5 μl of capillary blood added to 250 μl hemolysis reagent (Hengler Analytik) on a Olympus AU680 autoanalyzer device (Beckman Coulter) using an enzymatic UV test (hexokinase method) (Beckman Coulter). All animals were handled regularly and clinical signs were recorded at least twice a day (treatment day) and once a day (remaining study time). Animals were monitored carefully for any clinical signs of hypoglycemia, including behavior, coat, urine and fecal excretion, condition of body orifices and any sign of illness. In case of severe hypoglycemia food was offered or glucose solution was administered intravenous (i.v.), as required.

### In Vivo Cyno PK/PD

For the monkey experiments male cynomolgus monkeys (Macaca fascicularis) were acclimated after arrival (Nveprim Ltd, Mauritius) at least for 4 weeks. Animals were fed with NHP-Pellets (Ssniff, Germany) 200 gr per day and had free access to food and to controlled tap water ad libitum through automatic waterers. Animals were housed in groups in size and species-appropriate cages with a space of 260×200×220 cm. Monkeys were housed on a 12 h/12 light/dark cycle under controlled environmental conditions (temperature 20 to 24° C., relative humidity 40% to 75%). Animals were not fasted over night before compound administration. Iv administration was done using Venoflux microinfusers (23G) into the saphenous vein. After injection the remaining solution in the microinfuser was flushed with 0.9% NaCl. Sc compound administration was done using 26G needles directly in the intrascapular area. Blood was withdrawn from the saphenous vein using 2 ml syringes with 21 G needles. Animals were observed at least once daily and at each blood collection timepoint. During the time of the experiments no side effects were observed. Glucose concentration was determined from whole blood immediately after collection with an Accu-Check Active blood glucose meter (Roche Diagnostic). No hypoglycemia was observed.

### Scl-Fc-Detection in Plasma Using LC-MS/MS

50 μL of plasma sample were diluted with 250 μL PBS (Phosphate-Buffered Saline) containing IS (500 ng/ml) and immuno-enriched on the Thermo MSIA™ platform using MSIA Streptavidin D.A.R.T.'S tips. The analytes and Insulin-Standard (IS) were eluted with 65 μL of water/acetonitrile/TFA (66/33/0.4 v/v/v). After that, 100 UL of digestion buffer (100 mM of Ammonium bicarbonate, pH8.5), 2 L 0.1N sodium hydroxide and 3 μL DDT (dithiothreitol 500 mM in digestion buffer) were added into tube to incubate for 0.5 h at 60° C. on a ThermoMixer at 500 rpm. 5 L of IAA (iodoacetamide 500 mM in digestion buffer) was then added to tube, mixed, and set in the dark at room temperature for 45 min at 500 rpm. After that, 2 L DDT (dithiothreitol 500 mM in digestion buffer) were added into tube to incubate for 0.5 h at room temperature at 500 rpm. 10 μL of 100 μg/mL of TCPK-Trypsin (Thermo) in digestion buffer was added to tube to digest for 1.5 h on a ThermoMixer at 500 rpm and 37° C. The reaction was stopped by addition of 10 μL 10% formic acid in water, mixed and analyzed by LC-MS/MS.

### Scl-Fc-Detection in Plasma Using ELISA

MaxiSorp flat bottom plate (Nunc) plate was coated with anti-His-Tag monoclonal antibody (Novagen) at 1 μg/mL overnight at +4° C., washed three times using PBS/0.05% Tween-20 then blocked with 150 μL of 1% milk powder for 1 hour at room temperature (RT) and washed three times using PBS/0.05% Tween-20. The capture tool, 50 μL of recombinant insulin receptor (R&D Systems) was applied at 1.0 μg/mL in PBS/0.05% Tween-20, 50 μL/well, and incubated for 1 hour at RT, while shaking at 600 rpm. The plate was first washed three times with PBS/0.05% Tween-20. Then MRD diluted samples (inclusive standards, QCs and PK samples) were loaded on the plate and the plate was incubated at room temperature for 1.5 hours. Then the plate was washed three times with PBS/0.05% Tween-20 and the 1:20000 dilution of the HRP conjugated detection antibody (mouse anti-human IgG-Fc, Southern Biotech) in PBS/0.05% Tween-20 was applied, 100 μL/well, for 1 hour while shaking at 600 rpm to allow complexation between the detection tool and the scl. The plate was washed three times with PBS/0.05% Tween-20 and then the plate was incubated for 20 minutes with 100 μL of TMB, then 100 μL of stop solution (0.3 M HCl) were added. The OD signal was obtained with Infinity 1000 series of Tecan plate reader at 450 nm.

TABLE 1
Insulin receptor phosphorylation.

| | hIR-B- | | | hIR-A | | | Rat IR | | | Pig IR | | |
|---|---|---|---|---|---|---|---|---|---|---|---|---|
| | phosphorylation | | | phosphorylation | | | phosphorylation | | | phosphorylation | | |
| | [EC50 nM] | | | [EC50 nM] | | | [EC50 nM] | | | [EC 50 nM] | | |
| Variant | mean | SEM | n | mean | SEM | n | mean | SEM | n | mean | SEM | n |
| INS001 | 424 | 84 | 2 | 298 | 0 | 1 | 2320 | 0 | 1 | 59.8 | 0 | 1 |
| INS002 | 22.75 | 7.35 | 2 | 36 | 0 | 1 | 32.1 | 0 | 1 | 8.12 | 0 | 1 |
| INS003 | 67.4 | 23.8 | 2 | 82.2 | 32.9 | 3 | 51.4 | 6.7 | 3 | 13.95 | 4.85 | 2 |
| INS004 | 572.5 | 97.5 | 2 | 317.5 | 16.5 | 2 | 1277.5 | 952.5 | 2 | 368.5 | 265.5 | 2 |
| INS005 | 521.5 | 125.5 | 2 | 1510 | 0 | 1 | 1570 | 240 | 2 | 61.4 | 15.4 | 2 |
| INS006 | 387.5 | 158.5 | 2 | 1110 | 0 | 1 | 286 | 0 | 1 | 52.5 | 0 | 1 |
| INS007 | 57.3 | 3.5 | 2 | 82.45 | 39.6 | 2 | 54.8 | 12.2 | 2 | 10.8 | 0 | 1 |
| INS008 | 90 | 0 | 1 | 1410 | 0 | 1 | 440 | 0 | 1 | 21.2 | 0 | 1 |
| INS009 | 94.08 | 26.7 | 5 | 332 | 88.1 | 5 | 136.98 | 36.3 | 5 | 36.65 | 5.1 | 4 |
| INS010 | 501 | 260.2 | 3 | 1777 | 916.7 | 3 | 390 | 0 | 1 | 23.5 | 13.4 | 2 |
| INS011 | 109.5 | 21.4 | 6 | 413.8 | 77.6 | 5 | 168.52 | 38 | 5 | 39.64 | 12.1 | 5 |
| INS012 | 178 | 59 | 2 | 466 | 0 | 1 | 124.5 | 20.5 | 2 | 50.6 | 3.5 | 2 |
| INS013 | 57.2 | 0 | 1 | 5.58 | 0 | 1 | 23.6 | 0 | 1 | 4.17 | 0 | 1 |
| INS014 | 47.9 | 0 | 1 | 299.5 | 125.5 | 2 | 92.8 | 9.2 | 2 | 28.4 | 0 | 1 |
| INS015 | 56.3 | 5.1 | 2 | 211 | 29 | 2 | 95.65 | 0.1 | 2 | 33.5 | 2.3 | 2 |
| INS016 | 58.8 | 6.2 | 2 | 179.55 | 90.45 | 2 | 85.45 | 11.25 | 2 | 41.45 | 2.05 | 2 |
| INS017 | 36 | 4.6 | 3 | 70.6 | 8.5 | 2 | 53.65 | 3.35 | 2 | 26.3 | 2.5 | 3 |
| INS018 | 260.5 | 25.5 | 2 | 536 | 0 | 1 | 233 | 67 | 2 | 59.95 | 28.35 | 2 |
| INS019 | 292 | 79 | 2 | 666 | 0 | 1 | 2203.5 | 1786.5 | 2 | 43 | 2.3 | 2 |
| INS020 | 185 | 8 | 2 | 718.5 | 236.5 | 2 | 247 | 0 | 1 | 157.4 | 113.6 | 2 |
| INS021 | 287.5 | 90.5 | 2 | 447 | 0 | 1 | 620.5 | 439.5 | 2 | 56.7 | 0 | 1 |
| INS022 | 42.2 | 0 | 1 | 215.05 | 158.95 | 2 | 64.9 | 26.7 | 2 | 15.9 | 0 | 1 |
| INS023 | 59.8 | 33.2 | 2 | 408 | 29 | 2 | 381 | 208 | 2 | 29.9 | 7.3 | 2 |
| INS024 | 465.50 | 166.30 | 2 | 449.23 | 352.67 | 2 | 370.30 | 97.47 | 3 | 73.10 | 10.30 | 2 |
| Insulin | 12.67 | 0.88 | 58 | 18.62 | 1.34 | 39 | 21.55 | 1.86 | 35 | 8.02 | 0.44 | 33 |

TABLE 2
Pharmacokinetic parameters of selected scl-Fc variants in female Göttingen minipigs.

| Variant | INS003 | INS004 | INS005 | INS006 | INS007 | INS009 | INS011 | INS013 | INS018 | INS024 |
|---|---|---|---|---|---|---|---|---|---|---|
| Dosis/ | 6 nmol/ | 6 nmol/ | 6 nmol/ | 6 nmol/ | 3 nmol/ | 3 nmol/ | 6 nmol/ | 3 nmol/ | 3 nmol/ | 3 nmol/ |
| route of | kg sc | kg sc | kg sc | kg sc | kg sc | kg sc | kg sc | kg sc | kg sc | kg sc |
| administration | | | | | | | | | | |
| Detection | ELISA | ELISA | ELISA | ELISA | ELISA | ELISA | LC-MS/ | LC-MS/ | ELISA | LC-MS/ |
| method | (Ins) | (Ins) | (Ins) | (Ins) | (Ins) | (Ins) | MS | MS | (Ins) | MS |
| Tmax (h) | 24 | 61.3 | 14.7 | 21.3 | 33.3 | 34.7 | 14.7 | 42.7 | 57.3 | 25.3 |
| Cmax | 1550 | 2530 | 2290 | 1910 | 1780 | 2040 | 2840 | 1750 | 1990 | 1460 |
| (ng/mL) | | | | | | | | | | |
| Tlast(h) | 176 | 248 | 232 | 248 | 212 | 193 | 184 | 203 | 209 | 132 |
| AUClast | 152000 | 404000 | 241000 | 204000 | 189000 | 209000 | 238000 | 229000 | 272000 | 117000 |
| (h*ng/mL) | | | | | | | | | | |
| AUCinf (h | 191000 | 600000 | 267000 | 226000 | 226000 | 250000 | 309000 | 319000 | 366000 | 123000 |
| * ng/mL) | | | | | | | | | | |
| AUCextr (%) | 19.8 | 32.7 | 9.52 | 9.45 | 16.5 | 16.3 | 23.9 | 25.1 | 26.6 | 5.40 |
| t½ | 69.2 | 147 | 68.6 | 71.8 | 77.3 | 73.5 | 78.1 | 106 | 114 | 76 |
| MRT (h) | 112 | 227 | 103 | 108 | 124 | 114 | 103 | 158 | 167 | 63.7 |
| LLOQ | 100 | 100 | 100 | 100 | 100 | 100 | 50 | 100 | 100 | 50 |
| (ng/mL) | | | | | | | | | | |

PK Data in female Göttingen minipig
Mean plasma pharmacokinetic parameter (n = 3)
For calculation of mean concentrations, values below the lower limit of quantification were set to zero. The pharmacokinetic parameters were calculated by the program Phoenix WinNonlin 6.4 (Certara USA, Inc., Princeton, NJ) using a non-compartmental model and linear trapezoidal interpolation calculation.
Lower limit of quantification (LLOQ) as measured in plasma.
Tmax: Time of maximum observed drug plasma concentration
Cmax: Maximum observed plasma drug concentration
C0: Initial plasma drug concentration
Tlast: Time of last measurable concentration
AUClast: area under the plasma drug concentration-time curve from the time of dosing to the last measurable concentration
AUCinf: AUC from the time of doing to the last measurable concentration and extrapolated to infinity
AUCextr: Extrapolated AUC estimated as (AUCinf − AUClast/AUC inf)
T½: elimination time
CL: Plasma clearance
Vss: Apparent steady state volume of distribution
F(%): Bioavailability estimated as AUC inf ratio between sc and iv administration
MRT: Mean residence time

TABLE 3
Pharmacokinetic parameters of selected scl-Fc-variants in male cynomolgus monkey

| Variant | INS009 | INS009 | INS011 | INS011 | INS013 | INS013 |
|---|---|---|---|---|---|---|
| Dosis/route of | 1 nmol/kg | 1 nmol/kg | 1 nmol/kg | 1 nmol/kg | 1 nmol/kg | 1 nmol/kg |
| administration | iv | sc | iv | sc | iv | sc |
| Detection | LC- | LC- | LC- | LC- | LC- | LC- |
| method | MS/MS | MS/MS | MS/MS | MS/MS | MS/MS | MS/MS |
| Tmax (h) | | 24 | | 24 | | 24 |
| Cmax (ng/mL) | | 556 | | 838 | | 691 |
| C0 (ng/mL) | 1300 | | 2580 | | 3120 | |
| Tlast(h) | 176 | 209 | 131 | 336 | 209 | 203 |
| AUClast (h | 65600 | 55100 | 38900 | 83500 | 74800 | 58700 |
| *ng/mL) | | | | | | |
| AUCinf (h * | 75200 | 60000 | 42200 | 88600 | 79300 | 63600 |
| ng/mL) | | | | | | |
| AUCextr (%) | 11.5 | 8.15 | 7.91 | 4.67 | 5.74 | 7.59 |
| t½ | 59.1 | 57.3 | 40.1 | 69.9 | 51.4 | 50.1 |
| Cl(L/h/kg) | 0.00119 | | 0.00175 | | 0.000887 | |
| Vss(L/kg) | 0.0713 | | 0.0668 | | 0.0548 | |
| F(%) | 79.7 | | | | 80.2 | |

LLOQ = 50 ng/mL in plasma

### REFERENCES

Belfiore A, Malaguarnera R, Vella V, et al. Insulin Receptor Isoforms in Physiology and Disease: An Updated View. Endocr Rev. 2017; 38(5):379-431. doi:10.1210/er.2017-00073
Bright D J, Dunseath G J, Peter R, Luzio S. Stability of proinsulin in whole blood. Clin Biochem. 2018; 52:153-155. doi:10.1016/j.clinbiochem.2017.10.005
Bruhns P, Jönsson F. Mouse and human FcR effector functions. Immunol Rev. 2015; 268(1):25-51. doi:10.1111/imr.12350
Denley A, Carroll J M, Brierley G V, et al. Differential activation of insulin receptor substrates 1 and 2 by insulin-like growth factor-activated insulin receptors [published correction appears in Mol Cell Biol. 2007 September; 27(17): 6264]. Mol Cell Biol. 2007; 27(10):3569-3577. doi:10.1128/MCB.01447-06
Dodson G, Steiner D. The role of assembly in insulin's biosynthesis. Curr Opin Struct Biol. 1998; 8(2):189-194. doi:10.1016/s0959-440x(98)80037-7
Dumet C, Pottier J, Gouilleux-Gruart V, Watier H. Insights into the IgG heavy chain engineering patent landscape as applied to IgG4 antibody development [published correction appears in MAbs. 2019 November-December; 11(8):1]. MAbs. 2019; 11(8):1341-1350. doi:10.1080/19420862.2019.1664365
Faust C, Ochs C, Korn M, et al. Production of a novel heterodimeric two-chain insulin-Fc fusion protein. Protein Eng Des Sel. 2020; 33:gzaa026. doi:10.1093/protein/gzaa026
Glidden M D, Aldabbagh K, Phillips N B, et al. An ultra-stable single-chain insulin analog resists thermal inactivation and exhibits biological signaling duration equivalent to the native protein. J Biol Chem. 2018; 293(1):47-68. doi:10.1074/jbc.M117.808626
Godoy-Matos A F, Silva Júnior W S, Valerio C M. NAFLD as a continuum: from obesity to metabolic syndrome and diabetes. Diabetol Metab Syndr. 2020; 12:60. Published 2020 Jul. 14. doi:10.1186/s13098-020-00570-y
Hakuno F, Takahashi S I. IGF1 receptor signaling pathways. J Mol Endocrinol. 2018; 61(1):T69-T86. doi:10.1530/JME-17-0311
Hernández-Sánchez C, Mansilla A, de la Rosa E J, de Pablo F. Proinsulin in development: New roles for an ancient prohormone. Diabetologia. 2006; 49(6):1142-1150. doi:10.1007/s00125-006-0232-5
Hua Q X, Hu S Q, Jia W, et al. Mini-proinsulin and mini-IGF-I: homologous protein sequences encoding non-homologous structures. J Mol Biol. 1998; 277(1):103-118. doi:10.1006/jmbi.1997.1574
Jacobsen F W, Stevenson R, Li C, et al. Engineering an IgG Scaffold Lacking Effector Function with Optimized Developability. J Biol Chem. 2017; 292(5):1865-1875. doi:10.1074/jbc.M116.748525
Jehle P M, Lutz M P, Fussgaenger R D. High affinity binding sites for proinsulin in human IM-9 lymphoblasts. Diabetologia. 1996; 39(4):421-432. doi:10.1007/BF00400673
Kahn S E, Cooper M E, Del Prato S. Pathophysiology and treatment of type 2 diabetes: perspectives on the past, present, and future. Lancet. 2014; 383(9922):1068-1083. doi:10.1016/S0140-6736(13)62154-6
Landreh M, Johansson J, Jörnvall H. C-peptide: a molecule balancing insulin states in secretion and diabetes-associated depository conditions. Horm Metab Res. 2013; 45(11):769-773. doi:10.1055/s-0033-1347208
Mathieu C. Minimising hypoglycaemia in the real world: the challenge of insulin [published online ahead of print, 2021 Jan. 16]. Diabetologia. 2021; 10.1007/s00125-020-05354-7. doi:10.1007/s00125-020-05354-7
Munte C E, Vilela L, Kalbitzer H R, Garratt R C. Solution structure of human proinsulin C-peptide. FEBS J. 2005; 272(16):4284-4293. doi:10.1111/j.1742-4658.2005.04843.x
Rath T, Baker K, Dumont J A, et al. Fc-fusion proteins and FcRn: structural insights for longer-lasting and more effective therapeutics. Crit Rev Biotechnol. 2015; 35(2):235-254. doi:10.3109/07388551.2013.834293
Rosenstock J, Bajaj H S, Janež A, et al. Once-Weekly Insulin for Type 2 Diabetes without Previous Insulin Treatment. N Engl J Med. 2020; 383(22):2107-2116. doi:10.1056/NEJMoa2022474
Saltiel A R. Insulin signaling in health and disease. J Clin Invest. 2021; 131(1):e142241. doi:10.1172/JCI142241
Sand K M, Bern M, Nilsen J, Noordzij H T, Sandlie I, Andersen J T. Unraveling the Interaction between FcRn and Albumin: Opportunities for Design of Albumin-Based Therapeutics. Front Immunol. 2015; 5:682. Published 2015 Jan. 26. doi:10.3389/fimmu.2014.00682
Sapra A, Bhandari P. Diabetes Mellitus. In: StatPearls. Treasure Island (FL): StatPearls Publishing; Nov. 19, 2020.
Schlothauer T, Herter S, Koller C F, et al. Novel human IgG1 and IgG4 Fc-engineered antibodies with completely abolished immune effector functions. Protein Eng Des Sel. 2016; 29(10):457-466. doi:10.1093/protein/gzw040
Sharma A K, Taneja G, Kumar A, et al. Insulin analogs: Glimpse on contemporary facts and future prospective. Life Sci. 2019; 219:90-99. doi:10.1016/j.lfs.2019.01.011
Sommerfeld M R, Müller G, Tschank G, et al. In vitro metabolic and mitogenic signaling of insulin glargine and its metabolites. PLOS One. 2010; 5(3):e9540. Published 2010 Mar. 4. doi:10.1371/journal.pone.0009540
Steiner D F. On the discovery of precursor processing. Methods Mol Biol. 2011; 768:3-11. doi:10.1007/978-1-61779-204-5_1
Taylor S I, Yazdi Z S, Beitelshees A L. Pharmacological treatment of hyperglycemia in type 2 diabetes. J Clin Invest. 2021; 131(2):142243. doi:10.1172/JCI142243
Wang X, Mathieu M, Brezski R J. IgG Fc engineering to modulate antibody effector functions. Protein Cell. 2018; 9(1):63-73. doi:10.1007/s13238-017-0473-8
West E E, Kolev M, Kemper C. Complement and the Regulation of T Cell Responses. Annu Rev Immunol. 2018; 36:309-338. doi:10.1146/annurev-immunol-042617-053245
Yang Y, Hua Q X, Liu J, et al. Solution structure of proinsulin: connecting domain flexibility and prohormone processing. J Biol Chem. 2010; 285(11):7847-7851. doi:10.1074/jbc.C109.084921

1. An insulin-Fc conjugate of two insulin-Fc fusion polypeptides, wherein each of said two insulin-Fc fusion polypeptides comprises from N- to C-terminus: an insulin and an Fc region polypeptide, and
wherein said two insulin-Fc fusion polypeptides are linked by at least two covalent bonds at the C-terminus of the fusion polypeptides.

2. The insulin-Fc conjugate of claim 1, wherein the at least two covalent bonds are disulfide bonds.

3. The insulin-Fc conjugate of claim 1, wherein said conjugate is a homodimer of said two insulin-Fc fusion polypeptides and/or wherein the two insulin-Fc fusion polypeptides form an antibody Fc region.

4. The insulin-Fc conjugate of claim 1, wherein the insulin is a single chain insulin.

5. The insulin-Fc conjugate of claim 1, wherein each of the two insulin-Fc fusion polypeptides comprises from N- to C-terminus:
a) an insulin B chain,
b) a first linker peptide,
c) an insulin A chain,
d) a second linker peptide,
e) an Fc region polypeptide.

6. The insulin-Fc conjugate of claim 5, wherein each of the two insulin-Fc fusion polypeptides further comprises
f) a third linker peptide,
g) a C-terminal peptide allowing the formation of said at least two covalent bonds between said two insulin-Fc fusion polypeptides.

7. The insulin-Fc conjugate of claim 6, wherein the C-terminal peptide under g) comprises the sequence of an antibody hinge region comprising at least two cysteine residues allowing the formation of at least two disulfide bonds between the two insulin-Fc fusion polypeptides, for example wherein the sequence of the antibody hinge region comprises or consists of an amino acid sequence as shown in SEQ ID NO: 36, 39, 40 or 41.

8. The insulin-Fc conjugate of claim 1, wherein said two insulin-Fc fusion polypeptides are not covalently linked by one or more disulfide bonds between the N-terminus of each Fc region polypeptide and/or wherein the Fc region polypeptide lacks an antibody hinge region at the N-terminus.

9. The insulin-Fc conjugate of claim 1, wherein the Fc region polypeptide comprises constant domains of the heavy chain of an IgG, IgM, IgA, IgD or IgE antibody, for example wherein the Fc region polypeptide comprises the constant domains CH2 and CH3 of the antibody heavy chain an IgG1 antibody or an IgG4 antibody

10. The insulin-Fc conjugate of claim 1 wherein the first linker peptide has a length of 1 to 50, such as 5 to 15 amino acids, such as 5 to 10 amino acids, such as 7 amino acids, for example wherein the first linker peptide comprises or consists of the amino acid sequence GSYPGGV (SEQ ID NO: 26) or EEYPGDV (SEQ ID NO: 27), and/or
wherein said second linker peptide has a length of 1 to 100, such as of 5 to 40 amino acids, for example wherein said second linker peptide is the human insulin C-peptide, or a fragment thereof, for example wherein the said second linker comprises or consists of an amino acid sequence shown in SEQ ID NO: 28 or 29,
and/or
wherein said third linker has a length of 1 to 10 amino acids, such as a length of 6 amino acids, for example wherein said third linker peptide comprises or consists of an amino acid sequence as shown in GGGGSA (SEQ ID NO: 42).

11. The insulin-Fc conjugate of claim 1, wherein said insulin is an insulin having reduced binding affinity to the human insulin receptor as compared to the binding affinity of human insulin to human insulin receptor and/or wherein said insulin is an insulin shown in Table B, such as a single chain insulin shown in Table B, for example wherein
i) the insulin B chain comprises or consists of the amino acid sequence SFVNQHLCGSHLVEALELVCGERGFHYTPKT (SEQ ID NO: 69) and the insulin A chain comprises or consists of the amino acid sequence GIVEQCCTSICSLEQLENYCG (SEQ ID NO: 74),
ii) the insulin B chain comprises or consists of the amino acid sequence SFVNQHLCGSHLVEALHLVCGERGFAYTPKT (SEQ ID NO: 70) and the insulin A chain comprises or consists of the amino acid sequence GIVEQCCTSICSLEQLENYCG (SEQ ID NO: 74),
iii) the insulin B chain comprises or consists of the amino acid sequence FVNQHLCGSHLVEALHLVCGERGFHYTPKT (SEQ ID NO: 71) and the insulin A chain comprises or consists of the amino acid sequence GIVEQCCTSICSLSQLEDYCG (SEQ ID NO: 75),
iv) the insulin B chain comprises or consists of the amino acid sequence SFVNQHLCGSHLVEALELVCGERGFHYTPKT (SEQ ID NO: 73) and the insulin A chain comprises or consists of the amino acid sequence GIVEQCCTSICSLSQLEDYCG (SEQ ID NO: 75),
v) the insulin B chain comprises or consists of the amino acid sequence SFVNQHLCGSHLVEALYLVCGERGFFYTPKT (SEQ ID NO: 65) and the insulin A chain comprises or consists of the amino acid sequence GIVEQCCTSICSLEQLENYCG (SEQ ID NO: 74),
vi) the insulin B chain comprises or consists of the amino acid sequence SFVNQHLCGSHLVEALHLVCGERGFAYTDKT (SEQ ID NO: 66) and the insulin A chain comprises or consists of the amino acid sequence GIVEQCCTSICSLEQLENYCG (SEQ ID NO: 74), or
vii) the insulin B chain comprises or consists of the amino acid sequence SFVNQHLCGSHLVEALHLVCGERGFAYTDKT (SEQ ID NO: 67) and wherein the insulin A chain comprises or consists of the amino acid sequence GIVEQCCTSICSLEQLENYCG (SEQ ID NO: 74).

12. The insulin-Fc conjugate of claim 11, wherein said insulin is a single chain insulin comprising an amino acid sequence shown in SEQ ID NO: 50, SEQ ID NO: 52, SEQ ID NO: 53, SEQ ID NO: 58, SEQ ID NO: 45, SEQ ID NO: 47, SEQ ID NO: 81, or SEQ ID NO: 82.

13. The insulin-Fc conjugate of claim 11, wherein each of said two insulin-Fc fusion polypeptides comprises an amino acid sequence shown in SEQ ID NO: 10, SEQ ID NO: 12, SEQ ID NO: 14, SEQ ID NO: 19, SEQ ID NO: 4, SEQ ID NO: 5, SEQ ID NO: 6, or SEQ ID NO: 7.

14. An insulin according to the definition of the insulin in claim 11.

15. An insulin-Fc fusion polypeptide according to the definition of the insulin in claim 1.
